(19) United States
(12) Patent Application Publication
(10) Pub. No.: US 2017/0267991 A1
(43) Pub. Date: Sep. 21, 2017

(54) ACOUSTOPHORETIC CLARIFICATION OF PARTICLE-LADEN NON-FLOWING FLUIDS

(71) Applicant: FloDesign Sonics, Inc., Wilbraham, MA (US)

(72) Inventors: Bart Lipkens, Hampden, MA (US); Walter M. Presz, JR., Wilbraham, MA (US); Kedar Chitale, West Hartford, CT (US); Daniel Hatten, Wilbraham, MA (US); Nathaniel Bodnar, Watertown, CT (US); Goutam Ghoshal, Springfield, MA (US)

(21) Appl. No.: 15/588,472

(22) Filed: May 5, 2017

Related U.S. Application Data
(63) Continuation-in-part of application No. 14/870,952, filed on Sep. 30, 2015, now Pat. No. 9,675,906.
(60) Provisional application No. 62/332,111, filed on May 5, 2016.
(60) Provisional application No. 62/057,514, filed on Sep. 30, 2014.

(51) Int. Cl. C12N 13/00; B01D 21/26; C12N 1/02; B06B 1/06; C12N 5/078; C12N 5/0783; C12M 1/00; C12M 1/26; B01D 21/28; C12N 1/16

(52) U.S. Cl. CPC C12N 13/00; B01D 21/283; B01D 21/262; C12N 1/02; C12N 1/16; C12N 5/0641; C12N 5/0644; C12N 5/0634; C12N 5/0636; C12M 47/02; C12M 33/10; C12M 23/14; B06B 1/0622; B06B 2201/71

(57) ABSTRACT

Acoustophoretic devices for separating particles from a non-flowing host fluid are disclosed. The devices include a substantially acoustically transparent container and a separation unit, with the container being placed within the separation unit. An ultrasonic transducer in the separation unit creates a planar or multi-dimensional acoustic standing wave within the container, trapping particles disposed within the non-flowing fluid and causing them to coalesce or agglomerate, then separate due to buoyancy or gravity forces.

### CROSS-REFERENCE TO RELATED APPLICATIONS

This application claims priority to U.S. Provisional Patent Application Ser. No. 62/332,111, filed on May 5, 2016. This application is also a continuation-in-part of U.S. patent application Ser. No. 14/870,952, filed on Sep. 30, 2015, which claims priority to U.S. Provisional Patent Application Ser. No. 62/057,514, filed on Sep. 30, 2014. These applications are fully incorporated herein by reference in their entireties.

### BACKGROUND

Acoustophoresis is the separation of particles and secondary fluids from a primary or host fluid using high intensity acoustic standing waves, and without the use of membranes or physical size exclusion filters. It has been known that high intensity standing waves of sound can exert forces on particles in a fluid when there is a differential in both density and/or compressibility, otherwise known as the acoustic contrast factor. The pressure profile in a standing wave contains areas of local minimum pressure amplitudes at its nodes and local maxima at its anti-nodes. Depending on the density and compressibility of the particles, they will be trapped at the nodes or anti-nodes of the standing wave. The higher the frequency of the standing wave, the smaller the particles that can be trapped due to the pressure of the standing wave. The acoustophoretic process is typically performed on a moving fluid stream.

There are many applications for clarification of a fluid that contains particles or droplets, or separate the secondary phase from the host fluid. In certain situations it can be advantageous to execute such a process in a batch or semi-batch mode, especially when the concentration of the secondary phase is large, e.g., exceeding 1% by volume concentration, or e.g., exceeding 10%. Applications are in settling tanks, yeast separation processes in food and beverage industries, mammalian cell clarification in biopharmacy, and red and white blood cells from plasma.

Centrifuges are used for many biological processes to separate cells and other materials through gravitational separation of materials due to their density. The use of a centrifuge, however, may cause damage to some of the materials that are being separated in the process due to the high gravitational and shear forces that are experienced. This is especially true of biological materials such as cells. It is therefore desirable to find a different method of separating materials that does not involve high gravitational and high shear forces.

### BRIEF DESCRIPTION

The present disclosure relates, in various embodiments, to the use of ultrasonic energy in a standing wave to separate particles or secondary fluids in batch mode from a discrete volume of a fluid containing a mixture of a host fluid and particles/secondary fluid. The discrete volume of fluid is non-flowing, i.e. the fluid is not being pumped, flowed, or displaced by a second volume of fluid. Clarification of the discrete volume of fluid is accomplished using an acoustophoretic device. As a result, the discrete volume of fluid is separated into two portions, a portion with an increased concentration of particles and a portion with a decreased concentration of particles. The acoustophoresis separation process traps or suspends particles and droplets in their stable trapping locations within the acoustic field. A strong three-dimensional acoustic field further creates tightly packed clusters at these locations such that the gravity/buoyancy force becomes dominant, resulting in continuous settling of clusters or clumps of particles when they are heavier than the host liquid or rise out of suspension when the particles or droplets are lighter than the host fluid.

In various embodiments, the acoustophoretic device comprises a substantially acoustically transparent container having an upper end and a lower end; and a separation unit defined by one or more walls. The separation unit includes at least one ultrasonic transducer having a piezoelectric material driven by a signal to create an acoustic standing wave in the separation unit through the container, and the separation unit is separable from the container. The driving signal for the transducer may be based on voltage, current, magnetism, electromagnetism, capacitive or any other type of signal to which the transducer is responsive. The ultrasonic transducer can be driven to form a planar acoustic standing wave or a multi-dimensional acoustic standing wave.

Generally, the container holds the volume of fluid therein. The container is generally formed from a substantially acoustically transparent material, such as plastic, glass, polycarbonate, low-density polyethylene, and high-density polyethylene, having an appropriate thickness based on the frequency of the acoustic standing wave. The container may be a plastic bottle or plastic bag. In other embodiments, the container may be a centrifuge tube or a test tube.

In certain embodiments, the separation unit includes more than one ultrasonic transducer. The multiple, e.g., two, ultrasonic transducers may be located on a common wall of the separation unit. A single reflector may be used to reflect acoustic energy from/to one or more ultrasonic transducers, and/or may be used to propagate the standing wave in the separation unit. Alternatively, the ultrasonic transducers may be located opposite to each other, and be actuated to generate waves that cross one another.

In some embodiments, a wall of the separation unit includes a viewing window for viewing the separation occurring in the lower end of the container. The viewing window can further serve to allow the desired placement of the container in the separation unit.

Also disclosed herein is a method for clarifying a discrete volume of fluid medium containing particles using the container and separation unit previously described. The method generally comprises the steps of introducing the discrete volume of fluid medium to a container having an upper end and a lower end; placing the container into a separation unit defined by at least one wall, the separation unit including at least one ultrasonic transducer having a piezoelectric material capable of creating an acoustic standing wave in the separation unit by reflecting incident waves off of a reflector located opposite the at least one ultrasonic transducer; and driving the at least one ultrasonic transducer to create the acoustic standing wave in the separation unit to separate the particles from the discrete volume of fluid medium.

In some examples, driving the ultrasonic transducer to create the acoustic standing wave results in the creation of nodal lines and lateral forces that trap the particles of the discrete volume of fluid medium in those nodal lines. The particles in those nodal lines cluster, clump, agglomerate, or coalesce to form clusters that are large enough to sink to the lower end of the container due to gravitational forces or rise to the upper end of the container due to buoyancy forces. The sinking or rising of the clusters also creates a gravity-driven flow within the discrete volume itself, further enhancing the separation of the phases. In some embodiments, a fluid is interstitial between the container and the separation unit, such that the acoustic standing wave passes through the fluid in the separation unit and the discrete volume of fluid medium in the container.

In certain embodiments, the container may be a disposable separation bag including an exterior surface and an interior volume bounded by the exterior surface. In such embodiments, the ultrasonic transducer is at least partially disposed inward of the exterior surface of the separation bag such that an acoustic standing wave can be created in the interior volume of the bag. This allows for a disposable system whereby solids suspended within the fluid in the bag may be clumped, clustered, or agglomerated, and settle out of solution, and droplets emulsified in the fluid cluster, clump, agglomerate, or coalesce such that buoyancy forces the agglomerated or coalesced droplets to rise out of suspension. The acoustic standing wave field thus creates a clarification of the fluid in the bag. That is, in this arrangement, the cells, cell debris or other solids in the fluid are caught in the acoustic standing wave(s), clumped up into larger groups and fall back into the separation bag due to the force of gravity.

The ultrasonic transducer(s) of the present disclosure can generate an acoustic standing wave(s) having a frequency of from about 500 kHz to about 10 MHz.

Also disclosed herein are acoustic centrifuge systems for clarifying a discrete volume of fluid in each of a plurality of containers, the systems comprising a separation unit having an interstitial space therein. The separation unit comprises: a plurality of ultrasonic transducers arranged along a length of the separation unit, each transducer including a piezoelectric material configured to be driven to create an acoustic standing wave within the interstitial space; and an alignment plate along a top of the separation unit, the alignment plate having a plurality of apertures, each aperture being aligned with one of the ultrasonic transducers.

The separation unit may further comprise a transducer plate joining the plurality of ultrasonic transducers together, such that the plurality of ultrasonic transducers are arranged on a common wall of the separation unit.

The separation unit may further comprise a reflector plate joining a plurality of reflectors together, the reflector plate located across from the transducer plate on an opposite wall of the separation unit.

Each ultrasonic transducer may be a square transducer or a rectangular transducer. Each ultrasonic transducer may create a planar one-dimensional acoustic standing wave, or a multi-dimensional acoustic standing wave. The multi-dimensional acoustic standing wave may result in an acoustic radiation force having an axial force component and a lateral force component that are of the same order of magnitude.

The system may further comprise a container plate that moves relative to the separation unit. The container plate generally comprises a main portion and a flange portion, the main portion having a plurality of apertures therein, and the flange portion extending outwardly beyond the separation unit.

The system may further comprise an inlet and an outlet on the separation unit. These permit flowing a fluid through the interstitial space.

The system may further comprise a plurality of containers. These are inserted through the apertures, and can be for example test tubes or centrifuge tubes. Each container may have a square plan cross-section or a circular plan cross-section. Each container may be made of plastic, glass, polycarbonate, low-density polyethylene, or high-density polyethylene.

Also disclosed are methods for clarifying non-flowing fluid mixtures in a plurality of different containers, comprising: receiving a separation unit as described above; placing a plurality of substantially acoustically transparent containers through the apertures of the alignment plate, each container containing a non-flowing mixture of a host fluid and a second fluid or particulate; and driving each ultrasonic transducer to create the acoustic standing wave in a corresponding container, such that the second fluid or particulate in the corresponding container is trapped in the acoustic standing wave, clumps, clusters, agglomerates, or coalesces together, and continuously rises or settles out of the host fluid due to buoyancy or gravity forces.

The methods can further comprise moving the plurality of containers relative to the separation unit to sweep the non-flowing mixture from an upper end of the container to a lower end of the container. Alternatively, the methods can further comprise moving the plurality of containers relative to the separation unit to sweep the non-flowing mixture from a lower end of the container to an upper end of the container.

The plurality of substantially acoustically transparent containers may be placed through apertures in a container plate prior to placing the plurality of containers through the apertures of the alignment plate. The plurality of containers can then be moved relative to the separation unit by moving the container plate. The container plate may be moved using an automated device.

The frequency of the acoustic standing wave created by each transducer can be from about 500 kHz to about 10 MHz.

In particular embodiments, the host fluid is whole blood and the second fluid or particulate includes plasma and erythrocytes.

These and other non-limiting characteristics are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is a perspective view of one example embodiment of an acoustophoretic device of the present disclosure. A disposable container (e.g. a plastic bag) works in conjunction with a reusable separation unit containing one or more ultrasonic transducers.

FIG. 2 is a cross-sectional view of another example embodiment of an acoustophoretic device of the present disclosure, including a support structure for moving the separation unit relative to the container.

FIG. 3 is a perspective view of another example acoustophoretic device of the present disclosure with a support structure. Here, the separation unit contains the support structure within its walls, and the support structure only translates the ultrasonic transducer/reflector pair along an axis, while the walls of the separation unit remain in a static location relative to the container.

FIG. 4 is a cross-sectional diagram of a conventional ultrasonic transducer.

FIG. 5 is a cross-sectional diagram of an ultrasonic transducer of the present disclosure. An air gap is present within the transducer, and no backing layer or wear plate is present.

FIG. 6 is a cross-sectional diagram of an ultrasonic transducer of the present disclosure. An air gap is present within the transducer, and a backing layer and wear plate are present.

FIG. 7 is a graph showing the relationship of the acoustic radiation force, gravity/buoyancy force, and Stokes' drag force to particle size. The horizontal axis is in microns (μm) and the vertical axis is in Newtons (N).

FIG. 8 is a graph of electrical impedance amplitude versus frequency for a square transducer driven at different frequencies.

FIG. 9A illustrates the trapping line configurations for seven of the peak amplitudes of FIG. 8 from the direction orthogonal to fluid flow.

FIG. 9B is a perspective view illustrating the separator. The trapping lines are shown.

FIG. 9C is a view from the side of the separator, normal to the transducer of FIG. 9B, showing the trapping nodes of the standing wave where particles would be captured.

FIG. 9D is a view taken through the face of the transducer showing the trapping line configurations, along arrow 116 as shown in FIG. 9B.

FIG. 10 is a view of an acoustophoretic device of the present disclosure, showing a plastic bag (i.e. the container) partially disposed within a separation unit having an ultrasonic transducer driven by a voltage signal delivered by a BNC cable.

FIG. 11 is a schematic diagram of another example acoustophoretic device of the present disclosure. A disposable flexible plastic bag contains an embedded ultrasonic transducer.

FIG. 12 is a cross-sectional diagram of the bag of FIG. 11, showing the transducer embedded within the bag.

FIG. 13 illustrates an acoustic centrifuge process of the present disclosure depicting moving a test tube relative to an acoustic standing wave to sweep a non-flowing mixture from an upper end of the test tube to a upper end of the test tube.

FIG. 14 illustrates a first example embodiment of an acoustic centrifuge system of the present disclosure. In this example embodiment, the transducers do not span the height of the flow chamber.

FIG. 15 illustrates an exploded view of the components of the acoustic centrifuge system of FIG. 14.

FIG. 16 is an exploded view of a second example embodiment of an acoustic centrifuge system of the present disclosure. In this example embodiment, the transducers span substantially the height of the flow chamber.

FIG. 17 illustrates an acoustic centrifuge process of the present disclosure depicting moving a tube transversely relative to the separation unit of FIG. 16.

FIG. 18 is a photograph of an acoustic centrifuge system of the present disclosure constructed in accordance with the first example embodiment of FIG. 14.

FIG. 19 is a photograph of an acoustic centrifuge system of the present disclosure including a single ultrasonic transducer.

FIG. 20 illustrates an example embodiment of a centrifuge tube of the present disclosure in which blood has been separated into its constituent parts using an acoustic centrifuge process of the present disclosure.

FIG. 21A and FIG. 21B are photographs of an acoustic centrifuge tube containing a host fluid and yeast cells. FIG. 21A shows the acoustic centrifuge tube prior to being subjected to an acoustic centrifuge process. FIG. 21B shows the acoustic centrifuge tube after to being subjected to an acoustic centrifuge process.

FIG. 22 is a graph showing the pack cell mass (PCM) efficiency versus time for a 40% yeast solution subjected to an acoustic centrifuge process in the acoustic centrifuge system of FIG. 20. The y-axis is PCM efficiency (%) and runs from 0 to 80 in intervals of 10. The x-axis is time (minutes) and runs from 0 to 25 in intervals of 5.

FIG. 23 is a bar graph showing the effect of container shape on maximum PCM efficiency. The y-axis is maximum PCM efficiency (%) and runs from 65 to 79 in intervals of 2. The left bar is for a 4 mL square tube, and the right bar is for a 4 mL round tube.

FIG. 24 is a bar graph showing the effect of container material on maximum PCM efficiency. The y-axis is maximum PCM efficiency (%) and runs from 30 to 65 in intervals of 5. The left bar is for a 15 mL glass tube, and the right bar is for a 15 mL centrifuge tube (i.e. plastic).

FIG. 25 is a bar graph showing the effect of container volume on maximum PCM efficiency. The y-axis is maximum PCM efficiency (%) and runs from 0 to 100 in intervals of 20. The container volumes are, from left to right, 4 mL, 4 mL, 5 mL, 15 mL, 15 mL, and 50 mL.

FIG. 26A and FIG. 26B are two pictures showing a bag placed within a water bath in an acoustic chamber to cause separation of cells from fluid within the bag.

FIG. 27A and FIG. 27B are two pictures showing the bag of FIG. 26A, with the cells being concentrated into a cell pallet at the bottom of the bag, and fluid above the cell pallet. The bag itself is tapered.

FIG. 28A, FIG. 28B, and FIG. 28C are three pictures showing a separation unit that is in the form of a plastic bag with at least one ultrasonic transducer affixed to the surface thereof. A smaller bag is placed within the separation unit, the smaller bag containing fluid and cells to be separated. The pictures also show operation of the separation unit.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function. Furthermore, it should be understood that the drawings are not to scale.

The singular forms “a,” “an,” and “the” include plural referents unless the context clearly dictates otherwise.

The term “comprising” is used herein as requiring the presence of the named components/steps and allowing the presence of other components/steps. The term “comprising” should be construed to include the terms “consisting of” and “consisting essentially of”, which permit the presence of only the named components/steps and unavoidable impurities, and exclude other components/steps.

All numerical values used herein include values that are the same when reduced to the same number of significant figures and values that differ by less than the experimental error of conventional techniques for measuring that value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “substantially” and “about” can be used to include any numerical value that can vary without changing the basic function of that value. When used with a range, they also disclose the range defined by the absolute values of the two endpoints, e.g. “about 2 to about 4” also discloses the range “from 2 to 4.” The term “about” may refer to plus or minus 10% of the indicated number.

It should be noted that many of the terms used herein are relative terms. For example, the terms “upper” and “lower” are relative to each other in location, i.e. an upper component is located at a higher elevation than a lower component in a given orientation, but these terms can change if the device is flipped. The terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, i.e. the flow fluids through an upstream component prior to flowing through the downstream component. It should be noted that in a loop, a first component can be described as being both upstream of and downstream of a second component.

The terms “horizontal” and “vertical” are used to indicate direction relative to an absolute reference, i.e. ground level. However, these terms should not be construed to require structures to be absolutely parallel or absolutely perpendicular to each other. For example, a first vertical structure and a second vertical structure are not necessarily parallel to each other. The terms “top” and “bottom” are used to refer to surfaces or ends where the top is always higher than the bottom relative to an absolute reference, i.e. the surface of the earth. The terms “upwards” and “downwards” are also relative to an absolute reference; upwards is always against the gravity of the earth.

The term “parallel” should be construed in its lay sense of two surfaces that maintain a generally constant distance between them, and not in the strict mathematical sense that such surfaces will never intersect when extended to infinity.

The present application refers to “the same order of magnitude.” Two numbers are of the same order of magnitude if the quotient of the larger number divided by the smaller number is a value of at least 1 and less than 10.

The acoustophoretic separation technology of the present disclosure employs ultrasonic acoustic standing waves to trap particles or a secondary fluid in a volume of fluid containing said particles/secondary fluid. The particles or secondary fluid collect at the nodes or anti-nodes of the acoustic standing wave, depending on the particles' or secondary fluid's acoustic contrast factor relative to the host fluid, forming clusters/clumps/agglomerates/coalesced droplets that continuously fall out of the acoustic standing wave when the clusters have grown to a size large enough to overcome the holding force of the acoustic standing wave (e.g. by coalescence or agglomeration) and the particle/secondary fluid density is higher than the host fluid, or to rise out of the acoustic standing wave when the particle/secondary fluid density is less than the host fluid. The scattering of the acoustic field off the particles results in a three-dimensional acoustic radiation force, which acts as a three-dimensional trapping field. The three-dimensional acoustic radiation force generated in conjunction with an ultrasonic standing wave is referred to in the present disclosure as a three-dimensional or multi-dimensional standing wave. The acoustic radiation force is proportional to the particle volume (e.g. the cube of the radius) when the particle is small relative to the wavelength. It is proportional to frequency and the acoustic contrast factor. It also scales with acoustic energy (e.g. the square of the acoustic pressure amplitude). For harmonic excitation, the sinusoidal spatial variation of the force is what drives the particles to the stable axial positions within the standing waves. When the acoustic radiation force exerted on the particles is stronger than the combined effect of fluid drag force and buoyancy and gravitational force, the particle can be trapped within the acoustic standing wave field. This trapping results in concentration, agglomeration and/or coalescence of the trapped particles. The strong lateral forces create rapid clustering of particles. Micron-sized particles, e.g., bacteria, mammalian cells, micro-algae, metal particles, yeast, fungi, lipids, oil droplets, red blood cells, white blood cells, platelets, etc, can thus be separated from the host fluid through enhanced gravitational separation. For the case of a suspension with several different particle sizes, it is possible by tuning of the system parameters to settle out the group of particles that are larger in size whereas the group of particles smaller in size can be kept in suspension. These two layers can then be harvested separately. A repeated process can then be used to fractionate groups of different sized particles according to size.

In the acoustophoresis techniques discussed herein, the acoustic contrast factor is a function of the ratio of particle to fluid compressibility and particle to fluid density. Most cell types present a higher density and lower compressibility than the medium in which they are suspended, so that the acoustic contrast factor between the cells and the medium has a positive value. The axial acoustic radiation force (ARF) drives the cells, with a positive contrast factor, to the pressure nodal planes, whereas cells or other particles with a negative contrast factor are driven to the pressure anti-nodal planes. In some examples, radial or lateral components of the ARF are larger than the combined effect of fluid drag force and gravitational force. The radial or lateral component drives the cells/particles to specific locations (points) within these planes where they cluster, clump, agglomerate, or coalesce into larger groups, which will then continuously gravity separate from the fluid.

Desirably, the ultrasonic transducer(s) generate a three-dimensional or multi-dimensional acoustic standing wave in the fluid that exerts a lateral force on the suspended particles to accompany the axial force so as to increase the particle trapping and clumping capabilities of the standing wave. Typical results published in literature state that the lateral force is two orders of magnitude smaller than the axial force. In contrast, the technology disclosed in this application provides for a lateral force to be of the same order of magnitude as the axial force (i.e. a multi-dimensional acoustic standing wave). However, in certain embodiments described further herein, combinations of transducers that produce both multi-dimensional acoustic standing waves and planar standing waves are contemplated. For purposes of this disclosure, a standing wave where the lateral force is not the same order of magnitude as the axial force is considered a “planar acoustic standing wave.”

The multi-dimensional acoustic standing wave used for particle collection is obtained by driving an ultrasonic transducer at a frequency that both generates the acoustic standing wave and excites a fundamental 3D vibration mode of the piezoelectric material of the transducer. Perturbation of the piezoelectric element in an ultrasonic transducer in a multimode fashion allows for generation of a multi-dimensional acoustic standing wave. A piezoelectric element can be specifically designed to deform in a multimode fashion at designated frequencies, allowing for generation of a multi-dimensional acoustic standing wave. The multi-dimensional acoustic standing wave may be generated by distinct modes of the piezoelectric element such as the 3×3 mode that would generate multidimensional acoustic standing waves. A multitude of multidimensional acoustic standing waves may also be generated by allowing the piezoelectric element to vibrate through many different mode shapes. Thus, the piezoelectric element can be excited to generate multiple modes such as a 0x0 mode (i.e. a piston mode) to a 1x1, 2x2, 1x3, 3x1, 3x3, and other higher order modes and then cycle back through the lower modes of the piezoelectric element (not necessarily in straight order), or the excitation may be a weighted combination of several modes. This switching or dithering of the piezoelectric element between modes allows for various multidimensional wave shapes, along with a single piston mode shape to be generated over a designated time.

It is also possible to drive multiple ultrasonic transducers with arbitrary phasing and/or different or variable frequencies. Multiple transducers may work to separate materials in a fluid stream while being out of phase with each other and/or while operating at different or variable frequencies. Alternatively, or in addition, a single ultrasonic transducer that has been divided into an ordered array may also be operated such that some components of the array will be out of phase with other components of the array.

It may be desirable, at times, to modulate the frequency or voltage amplitude of the standing wave. Such modulation may be done by amplitude modulation and/or by frequency modulation of the drive signal provided to the acoustic transducer. The duty cycle of the propagation of the standing wave may also be utilized to achieve certain results for trapping of materials. The acoustic beam may be turned on and shut off, at the same or different frequencies, to achieve desired results.

The lateral force of the total acoustic radiation force (ARF) generated by the ultrasonic transducers of the present disclosure is significant and is sufficient to overcome the fluid drag force at high linear velocities up to 1 cm/s and beyond. For example, linear velocities through the devices of the present disclosure can be a minimum of 4 cm/min for separation of cells/particles, and can be as high as 1 cm/sec for separation of oil/water phases. This can be relevant when, as described further below, an ultrasonic transducer is moved relative to a standing volume of fluid to enhance separation.

If desired, multiple standing waves from multiple ultrasonic transducers can also be used, which allows for multiple separation stages. For example, in a mixture of particles and fluid, the first transducer (and its standing wave) collects a certain amount of the particles, and the second transducer (and its standing wave) collects additional particles that passed through the first transducer. This construction can be useful where the particle/fluid ratio is high (i.e. large volume of particles), and the separation capacity of the first transducer is reached. This construction can also be useful for particles that have a bimodal or greater size distribution, where each transducer can be implemented to capture particles within a certain size range.

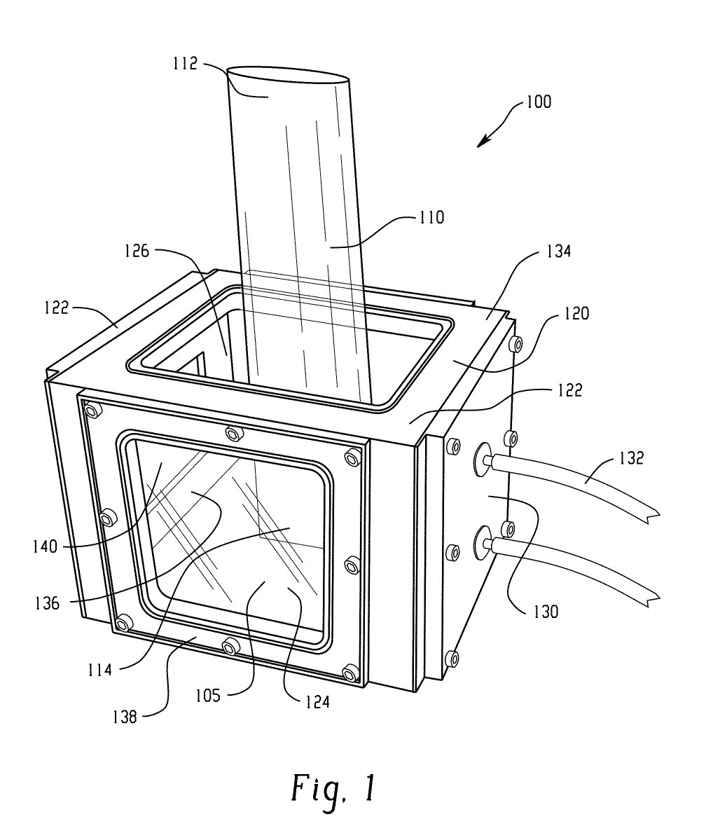

FIG. 1 illustrates a first example embodiment of an acoustophoretic device 100 of the present disclosure to be used with a discrete volume of fluid medium. This fluid may be considered to be non-flowing, in that there is no pump moving the fluid, and there is no additional fluid being added to this discrete volume or displaced from this discrete volume. The acoustophoretic device 100 includes a substantially acoustically transparent container 110 and a separation unit 120. Container 110 and separation unit 120 are separable from each other.

The container 110 of the acoustophoretic device 100 is formed from a substantially acoustically transparent material such as plastic, glass, polycarbonate, low-density polyethylene, and high-density polyethylene (all at an appropriate thickness). However, the container 110 may be formed from any material suitable for allowing the passage of the acoustic standing wave(s) of the present disclosure therethrough. The container 110 may be in the form of a bottle or a bag or an enclosure that fits in the separation unit 120. The difference between these forms lies in their composition and structure. A bottle is more rigid than a bag. When empty, some bags do not support themselves in a stable shape, while a bottle is able to stand upright. For example, container 110 as shown in FIG. 1 is a high-density polyethylene bag. Container 110 has an upper end 112 and a lower end 114, and an interior volume in which the non-flowing fluid medium is located. This fluid medium is a mixture of a host fluid which is a majority of the fluid medium, and a second fluid or particulate which is dispersed in the host fluid.

The separation unit 120 of the acoustophoretic device is defined by at least one wall 122, and may have a plurality of walls, which form the sides of the separation unit 120. For example, the separation unit 120 may be in the shape of a cylinder, or a rectangle, as depicted in FIG. 1. The wall(s) of separation unit 120 are solid and may be rigid or flexible. An opening 126 is present in an upper end of the separation unit, for receiving the container 110 therethrough. As the separation unit 120 is separable from the container 110, the container can be disposable or reusable, depending upon the desired application of the acoustophoretic device. As illustrated in FIG. 1, the base of the separation unit 120 is solid, and may be rigid or flexible.

The separation unit 120 includes at least one ultrasonic transducer 130 on a wall 134. The ultrasonic transducer 130 has a piezoelectric element that can be driven by a voltage signal to create an acoustic standing wave. The ultrasonic transducer(s) may be driven by an electrical signal, which may be controlled based on voltage, current, phase angle, power, frequency or any other electrical signal characteristic. Cables 132 are illustrated for transmitting power and control information to the ultrasonic transducer 130. A reflector 140 is located on the wall 136 opposite the ultrasonic transducer 130. The standing wave is generated through initial waves radiated from the transducer 130 upon be actuated, in combination with reflected waves from the reflector 140. In some embodiments, a reflector may be omitted. Ambient air may be used as a pressure release boundary to reflect the incident waves and create the standing waves. Various transducer and reflector combinations may be utilized for the creation of the acoustic standing wave(s) of the present disclosure to accelerate the gravity settling of particles or the buoyancy rising of particles or low-density fluids, respectively, that are disposed within the non-flowing fluid medium. The planar and/or multi-dimensional acoustic standing wave(s) are generated within the container, and are used to increase the speed of settling of particles in a non-flowing fluid in the container. This process may also be utilized in a batch or semi-batch operation where the particle and fluid mixture may be stopped for a period of time while the acoustic standing wave is used to accelerate the separation of particles in the fluid before resuming introduction of the fluid with the particles now separated to the bottom of a well or catch area. In some examples, there is no contact between the ultrasonic transducer and the discrete volume of fluid that is being separated.

In certain embodiments, the acoustophoretic device includes a plurality of ultrasonic transducers 130 located on a common wall 134 of the separation unit opposite the wall 136 on which the reflector 140 is located. Alternatively, or in addition, the ultrasonic transducers can be located opposite each other, with no reflector being present, or with an ultrasonic transducer acting as a reflector. The separation unit 120 includes a viewing window 124 in another wall 138 of the separation unit 120. As shown in the embodiment of FIG. 1, viewing window 124 is in a wall of the separation unit 120 adjacent the walls upon which the ultrasonic transducer(s) 130 and reflector 140 are located, such that the lower end 114 of the container 110 can be viewed through the viewing window 124 in the separation chamber 120. In other embodiments, the viewing window 124 can take the place of the reflector 140.

In certain embodiments, a fluid, such as water, may be placed in the interstitial space 105 between the container 110 and the separation unit 120, such that the acoustic standing wave passes through both the fluid in the separation unit 120 and the non-flowing fluid medium in the container 110. The interstitial fluid can be any fluid, though it is desirable to use fluid with acoustic properties similar to the discrete volume of fluid in the container 110, so as not to prevent the acoustic standing wave(s) from passing through the non-flowing fluid medium in the container 110 for separation and clarification therein. The fluid in the interstitial space should have an acoustic impedance value that allows for good transmission of the acoustic standing wave(s), and preferably a low acoustic attenuation.

In certain embodiments, the separation unit 120 includes a support structure that is configured to move the ultrasonic transducer(s) 130 vertically relative to the container 110, along with the reflector 140 when present. The movement of the transducer creates a “sweeping effect” through the non-flowing fluid mixture in the container 110 from the upper end 112 to the lower end 114 or from the lower end 114 to the upper end 112 thereof, depending on the direction of the vertical movement. This “sweeping” of the fluid in the container improves the settling or buoyancy of particles that are disposed within the fluid. The ultrasonic transducer 130 may be moved relative to the container at a linear velocity of from about 0.1 millimeter/second to about 1 centimeter/second. Although acoustophoretic device 100 is described and shown to have ultrasonic transducer 130 move vertically along container 110, other implementations are possible. For example, ultrasonic transducer 130 and container 110 can both be moved relative to each other. Ultrasonic transducer 130 can be fixed in location and container 110 can be moved. The movement of ultrasonic transducer 130 and/or container 110 can be in any particular direction with respect to gravity, e.g. horizontally or at an angle between vertical and horizontally. The relative movement can be rotational, e.g. ultrasonic transducer 130 can rotate around container 110. According to another example, one or more ultrasonic transducers 130 can be provided along a direction of settling, e.g. in an array, and actuated in series. In such an arrangement, the array of ultrasonic transducers and the container 110 need not move with respect to each other. In some examples, ultrasonic transducer 130 occupies a substantial area with respect to container 110, and relative movement can be omitted.

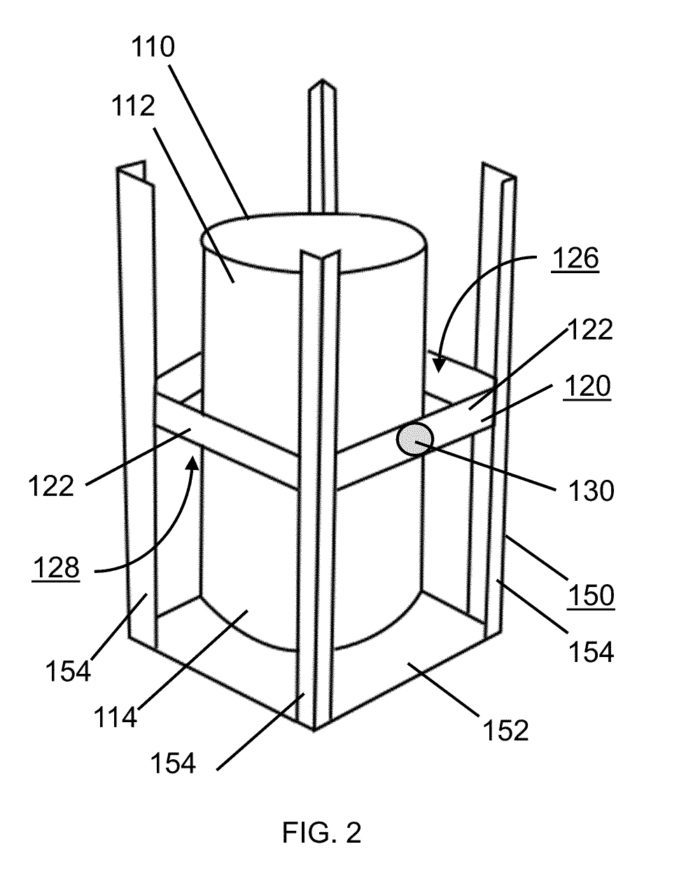

An example acoustophoretic device, such as may be implemented as acoustophoretic device 100, is depicted in FIG. 2. Here, the separation unit 120 is made up of four walls 122, with the transducer 130 on one of the walls (reflector not visible). The unit 120 includes an upper opening 126 and a lower opening 128, with the container 110 passing through both openings. The support structure 150 here includes a base 152 and support pillars 154 rising vertically from the base. The support pillars provide a mechanism for moving the separation unit 120 up and down relative to the container 110, which maintains its position on the base. The mechanism can be any known in the art, e.g. gears, pulleys, etc. The separation unit 120 is depicted here at roughly the middle of the container 110, and arrows indicate that the separation unit 120 can move upwards or downwards as desired. For example, the transducer 130 can be moved from the upper end 112 towards the lower end 114 of the container 110 so as to enhance the settling of the particles at the bottom of the container. Alternatively, the transducer 130 could be raised from the lower end 114 of the container towards the upper end 112 of the container 110 so as to increase the separation of buoyant particles, such as in an oil-water mixture where the oil is being separated from the water.

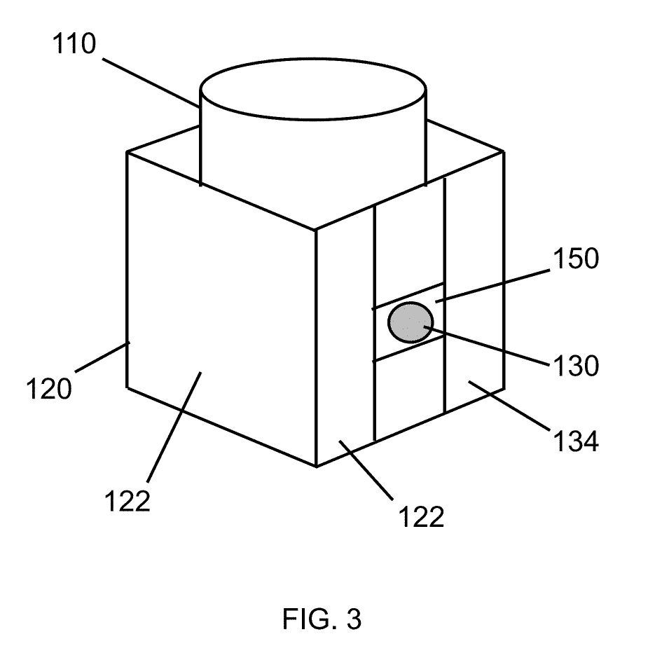

Another embodiment of such a device is present in FIG. 3. Here, the ultrasonic transducer 130 is mounted on a support structure 150 within the separation unit 120, and the wall 134 includes a track along which the support structure moves vertically relative to the container 110. The walls 122 of the separation unit 120 do not move relative to the container 110, only the transducer (and reflector if present).

The various parts of the acoustophoretic devices of this disclosure can be made from any suitable material. Such suitable materials include medical grade plastics, such as polycarbonates or polymethyl methacrylates, or other acrylates, metals such as steel, or glass. It is generally desirable for the material to be somewhat transparent, so that a clear window can be produced and the internal flow channels and flow paths can be seen during operation of the acoustophoresis device/system.

Some explanation of the ultrasonic transducers used in the devices of the present disclosure may be helpful as well. In this regard, the transducers use a piezoelectric element, usually made of PZT-8 (lead zirconate titanate). Such elements may have a 1 inch diameter and a nominal 2 MHz resonance frequency, or may be of a square or rectangular shape. Each ultrasonic transducer module can have only one piezoelectric element, or can have multiple piezoelectric elements that each act as a separate ultrasonic transducer and are either controlled by one or multiple amplifiers.

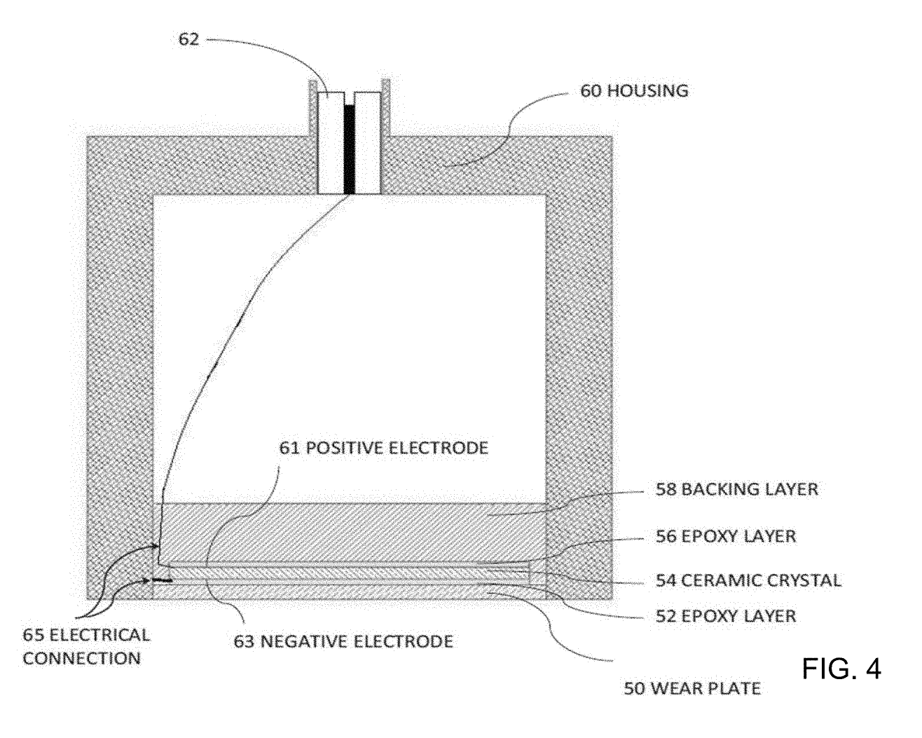

FIG. 4 is a cross-sectional diagram of a conventional ultrasonic transducer. This transducer has a wear plate 50 at a bottom end, epoxy layer 52, ceramic piezoelectric element 54 (made of, e.g. PZT), an epoxy layer 56, and a backing layer 58. On either side of the ceramic piezoelectric element, there is an electrode: a positive electrode 61 and a negative electrode 63. The epoxy layer 56 attaches backing layer 58 to the piezoelectric element 54. The entire assembly is contained in a housing 60 which may be made out of, for example, aluminum. An electrical adapter 62 provides a connection for wires to pass through the housing and connect to leads (not shown) which attach to the piezoelectric element 54. Typically, backing layers are designed to add damping and to create a broadband transducer with uniform displacement across a wide range of frequency and are designed to suppress excitation at particular vibrational eigen-modes. Wear plates are usually designed as impedance transformers to better match the characteristic impedance of the medium into which the transducer radiates.

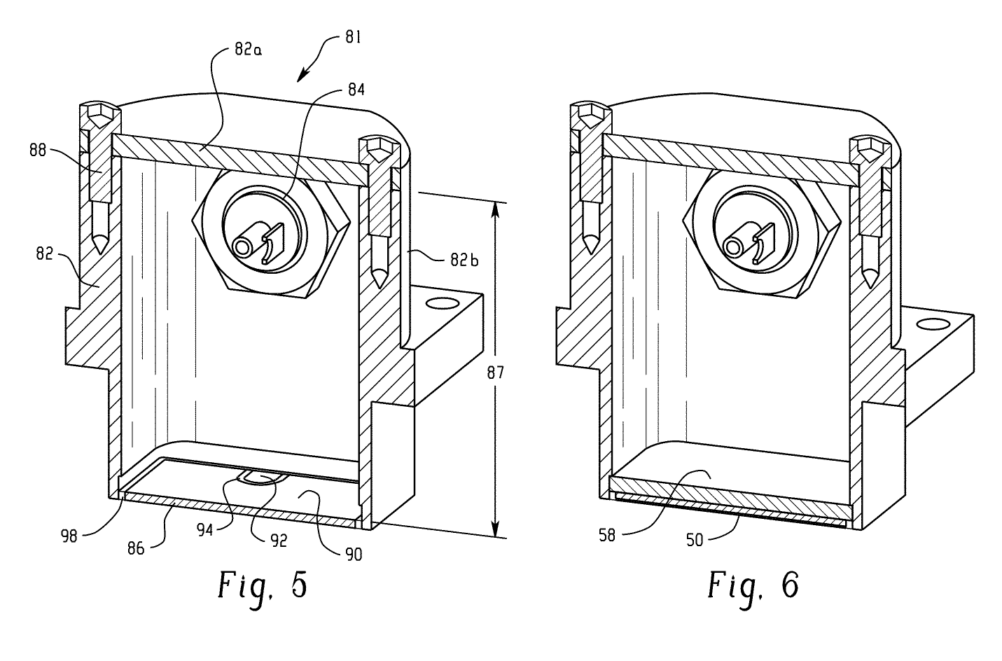

FIG. 5 is a cross-sectional view of an ultrasonic transducer 81 of the present disclosure. Transducer 81 is shaped as a square, and has an aluminum housing 82. The piezoelectric element is a mass of perovskite ceramic, each consisting of a small, tetravalent metal ion, usually titanium or zirconium, in a lattice of larger, divalent metal ions, usually lead or barium, and O²⁻ ions. As an example, a PZT (lead zirconate titanate) piezoelectric element 86 defines the bottom end of the transducer, and is exposed at the exterior of the bottom end of the housing. The piezoelectric element is supported on its perimeter by a small elastic layer 98, e.g. epoxy, silicone or similar material, located between the piezoelectric element and the housing. Transducer 81 does not include a wear plate or backing material. In some embodiments, a layer of plastic or other material (not shown) is provided over the exterior surface of piezoelectric element 86. The material, has a feature of separating piezoelectric element 86 from the fluid in which the acoustic standing wave is being generated. The material may be relatively thin, e.g. in the range of 10 μm-1 mm, and may be fastened to piezoelectric element 86 with adhesive, for example. The material may be substantially acoustically transparent, as may be the adhesive. Piezoelectric element 86 has an exterior surface (which is exposed) and an interior surface.

Screws 88 attach an aluminum top plate 82a of the housing to the body 82b of the housing via threads. The top plate includes a connector 84 for powering the transducer. The top surface of the PZT piezoelectric element 86 is connected to a positive electrode 90 and a negative electrode 92, which are separated by an insulating material 94. The polarities of electrodes 90, 92 can be reversed, and electrodes 90, 92 can be located on opposing sides or the same side of piezoelectric element 86, where the same side can be an interior surface or an exterior surface. The electrodes can be made from any conductive material, such as silver or nickel. Electrical power is provided to the PZT piezoelectric element 86 through the electrodes on the piezoelectric element. Note that the piezoelectric element 86 has no backing layer or epoxy layer. Put another way, there is an air gap 87 in the transducer between aluminum top plate 82a and the piezoelectric element 86 (i.e. e.g., the housing is empty or contains atmospheric air). A minimal backing 58 and/or wear plate 50 may be provided in some embodiments, as seen in FIG. 6.

The transducer design can affect performance of the system. A typical transducer is a layered structure with the ceramic piezoelectric element bonded to a backing layer and a wear plate. Because the transducer is loaded with the high mechanical impedance presented by the standing wave, the traditional design guidelines for wear plates, e.g., half wavelength thickness for standing wave applications or quarter wavelength thickness for radiation applications, and manufacturing methods may not be appropriate. In the example of transducer 81, illustrated in FIG. 5, there is no wear plate or backing, allowing piezoelectric element 86 to vibrate in one of its eigenmodes with a high Q-factor, or in a combination of several eigenmodes. Piezoelectric element 86 has an exterior surface that is directly exposed to the fluid flowing through a flow chamber to which transducer 81 is mounted.

Removing the backing (e.g. making the piezoelectric element air backed) also permits the ceramic piezoelectric element to vibrate at higher order modes of vibration with little damping (e.g. higher order modal displacement). In a transducer having a piezoelectric element with a backing as previously implemented, the piezoelectric element vibrates with a more uniform displacement, like a piston. Removing the backing allows the piezoelectric element to more readily vibrate in a non-uniform displacement mode. The higher order the mode shape of the piezoelectric element, the more nodal lines the piezoelectric element can generate. The higher order modal displacement of the piezoelectric element creates more trapping lines, although the correlation of trapping line to node may not necessarily be one to one, and driving the piezoelectric element at a higher or lower frequency may not necessarily produce more or less trapping lines for a given frequency of operation.

In some embodiments, the piezoelectric element may have a backing that has a relatively small effect on the Q-factor of the piezoelectric element (e.g. less than 5%). The backing may be made of a substantially acoustically transparent material such as balsa wood, foam, or cork which allows the piezoelectric element to vibrate in a higher order mode shape and maintains a high Q-factor while still providing some mechanical support for the piezoelectric element. The backing layer may be a solid, or may be a lattice having holes through the layer, such that the lattice follows the nodes of the vibrating piezoelectric element in a particular higher order vibration mode, providing support at node locations while allowing the rest of the piezoelectric element to vibrate freely. The goal of the lattice work or acoustically transparent material is to provide support without lowering the Q-factor of the piezoelectric element or interfering with the excitation of a particular mode shape.

The transducer can be driven by a drive signal, such as a voltage signal, a current signal, a magnetic signal, an electromagnetic signal, a capacitive signal, or any other type of signal to which the transducer is responsive to create a multi-dimensional acoustic standing wave. In embodiments, the voltage signal driving the transducer can have a pulsed, sinusoidal, square, sawtooth, or triangle waveform; and have a frequency of 500 kHz to 10 MHz. The voltage signal can be driven with pulse width modulation, which can be used to produce any desired waveform. The voltage signal can be amplitude or frequency modulated. The drive signal may be turned on or off and/or configured with start/stop capability to, for example, eliminate streaming.

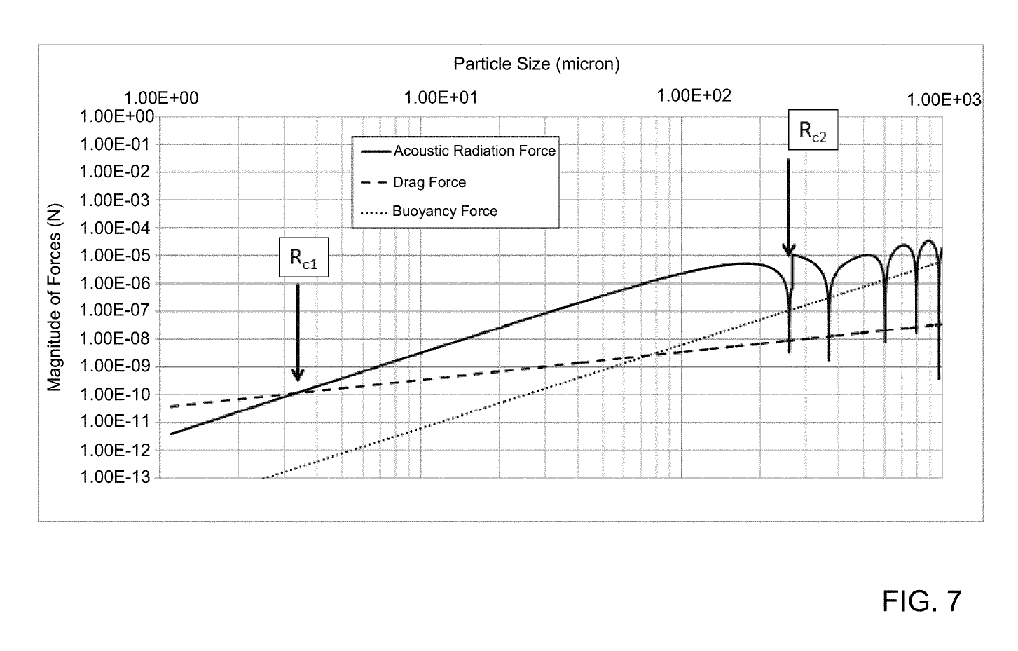

FIG. 7 is a lin-log graph (linear y-axis, logarithmic x-axis) that shows the calculated scaling of the acoustic radiation force, fluid drag force, and buoyancy force with particle radius. The buoyancy force is applicable to negative contrast factor particles, such as oil particles in this example. The calculated buoyancy force may include elements of gravity forces. In examples using positive contrast factor particles, which may be some types of cells, a line indicating gravity forces is used in a graph for such positive contrast factor particles showing acoustic radiation force and fluid drag force. In the present example illustrated in FIG. 7 calculations are done for a typical SAE-30 oil droplet used in experiments. The buoyancy force is a particle volume dependent force, e.g., proportional to the radius cubed, and is relatively negligible for particle sizes on the order of a micron, but grows, and becomes significant for particle sizes on the order of hundreds of microns. The fluid drag force scales linearly with fluid velocity, e.g., proportional to the radius squared, and typically exceeds the buoyancy force for micron sized particles, but is less influential for larger sized particles on the order of hundreds of microns. The acoustic radiation force scaling acts differently than the fluid drag force or the buoyancy force. When the particle size is small, the acoustic trapping force scales with the cube of the particle radius (volume) of the particle at a close to linear rate. Eventually, as the particle size grows, the acoustic radiation force no longer increases linearly with the cube of the particle radius. As the particle size continues to increase, the acoustic radiation force rapidly diminishes and, at a certain critical particle size, is a local minimum. For further increases of particle size, the radiation force increases again in magnitude but with opposite phase (not shown in the graph). This pattern repeats for increasing particle sizes. The particle size to acoustic radiation force relationship is at least partially dependent on the wavelength or frequency of the acoustic standing wave. For example, as a particle increases to a half-wavelength size, the acoustic radiation force on the particle decreases. As a particle size increases to greater than a half-wavelength and less than a full wavelength, the acoustic radiation force on the particle increases.

Initially, when a suspension is flowing through the acoustic standing wave with primarily small micron sized particles, the acoustic radiation force balances the combined effect of fluid drag force and buoyancy force to trap a particle in the standing wave. In FIG. 7, trapping occurs for a particle size of about 3.5 micron, labeled as Rc1. In accordance with the graph in FIG. 7, as the particle size continues to increase beyond Rc1, larger particles are trapped, as the acoustic radiation force increases compared to the fluid drag force. As small particles are trapped in the standing wave, particle coalescence/clumping/aggregation/agglomeration takes place, resulting in continuous growth of effective particle size. Other, smaller particles continue to be driven to trapping sites in the standing wave as the larger particles are held and grow in size, contributing to continuous trapping. As the particle size grows, the acoustic radiation force on the particle increases, until a first region of particle size is reached. As the particle size increases beyond the first region, the acoustic radiation force on the particle begins to decrease. As particle size growth continues, the acoustic radiation force decreases rapidly, until the buoyancy force becomes dominant, which is indicated by a second critical particle size, Rc2, at which size the particles rise or sink, depending on their relative density or acoustic contrast factor with respect to the host fluid. As the particles rise or sink and leave the antinode (in the case of negative contrast factor) or node (in the case of positive contrast factor) of the acoustic standing wave, the acoustic radiation force on the particles may diminish to a negligible amount. The acoustic radiation force continues to trap small and large particles, and drive the trapped particles to a trapping site, which is located at a pressure antinode in this example. The smaller particle sizes experience a reduced acoustic radiation force, which, for example, decreases to that indicated near point Rc1. As other particles are trapped and coalesce, clump, aggregate, agglomerate and/or cluster together at the node or antinode of the acoustic standing wave, effectively increasing the particle size, the acoustic radiation force increases and the cycle repeats. All of the particles may not drop out of the acoustic standing wave, and those remaining particles may continue to grow in size. Thus, FIG. 7 explains how small particles can be trapped continuously in a standing wave, grow into larger particles or clumps, and then eventually rise or settle out because of the relationship between buoyancy force, drag force and acoustic radiation force with respect to particle size.

In some examples, the size, shape, and thickness of the transducer can determine the transducer displacement at different frequencies of excitation. Transducer displacement with different frequencies may affect separation efficiency. In some examples, the transducer is operated at frequencies near the thickness resonance frequency (half wavelength). The presence of gradients in transducer displacement can result in more places for particles to be trapped. Higher order modal displacements can generate three-dimensional acoustic standing waves with strong gradients in the acoustic field in all directions, thereby creating similarly strong acoustic radiation forces in all directions, which forces may, for example, be on the same order of magnitude. The higher order modal displacements can lead to multiple trapping lines. The number of trapping lines correlate with the particular mode shape of the transducer.

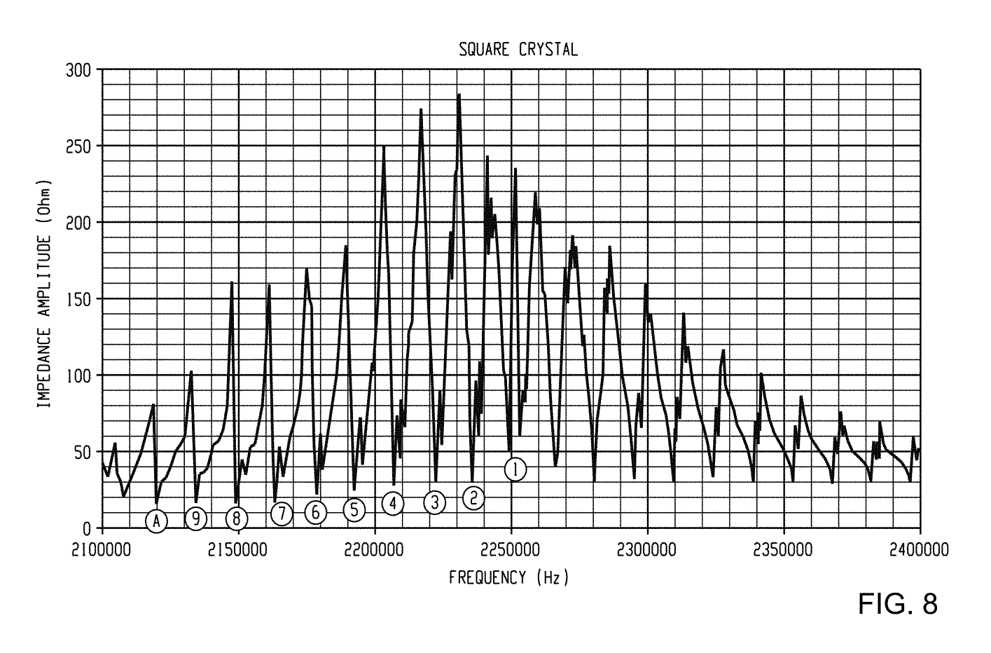

FIG. 8 shows the measured electrical impedance amplitude of the transducer as a function of frequency in the vicinity of the 2.2 MHz transducer resonance. The minima in the transducer electrical impedance correspond to acoustic resonances of a water column and represent potential frequencies for operation. Numerical modeling has indicated that the transducer displacement profile varies significantly at these acoustic resonance frequencies, and thereby directly affects the acoustic standing wave and resulting trapping force. Since the transducer may be operated near its thickness resonance, the displacements of the electrode surfaces are essentially out of phase. The displacement of the transducer electrodes may not be uniform and varies depending on frequency of excitation. As an example, at one frequency of excitation with a single line of trapped oil droplets, the displacement has a single maximum in the middle of the electrode and minima near the transducer edges. At another excitation frequency, the transducer profile has multiple maxima leading to multiple trapped lines of oil droplets. Higher order transducer displacement patterns can result in higher trapping forces and multiple stable trapping lines for the captured oil droplets.

To investigate the effect of the transducer displacement profile on acoustic trapping force and particle separation efficiencies, an experiment was repeated ten times, with all conditions identical except for the excitation frequency. Ten consecutive acoustic resonance frequencies, indicated by circled numbers 1-9 and letter A on FIG. 8, were used as excitation frequencies. The conditions were experiment duration of 30 min, a 1000 ppm oil concentration of approximately 5-micron SAE-30 oil droplets, a flow rate of 500 ml/min, and an applied power of 20 W.

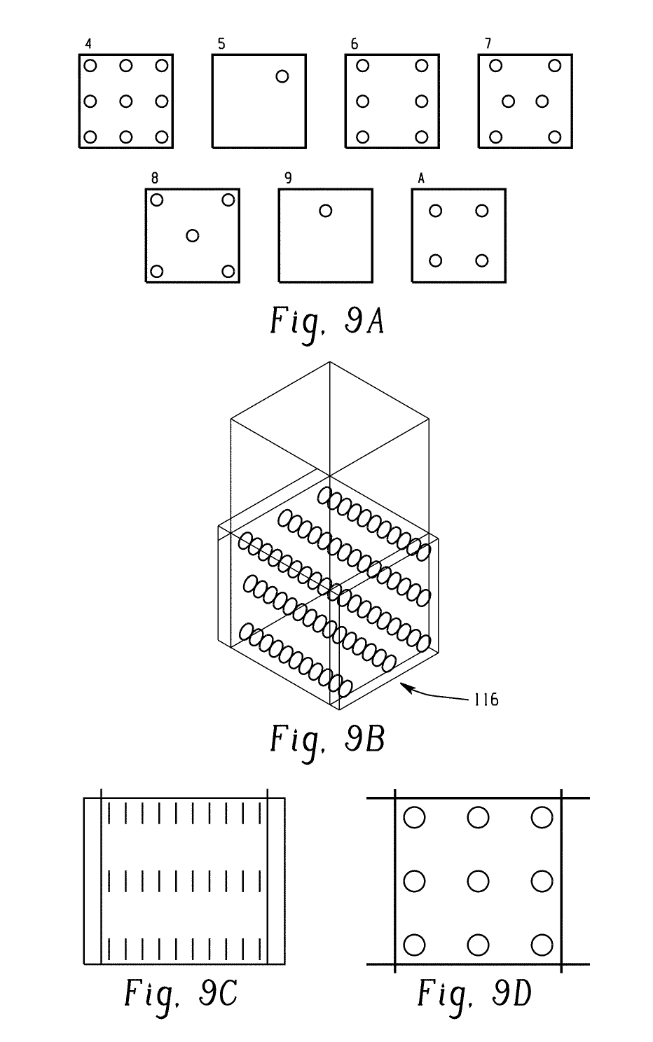

As the emulsion passed by the transducer, the trapping lines of oil droplets were observed and characterized. The characterization involved the observation and pattern of the number of trapping lines across the fluid channel, as shown in FIG. 9A, for seven of the ten resonance frequencies identified in FIG. 8.

FIG. 9B shows an isometric view of a system of the present disclosure where the trapping line locations are indicated. FIG. 9C is a view of the system as it appears from the side, looking at the trapping lines. FIG. 9D is a view of the system as it appears when looking directly at the transducer face, along arrow 116.

The effect of excitation frequency in this example clearly determines the number of trapping lines, which vary from a single trapping line at the excitation frequency of acoustic resonance 5 and 9, to nine trapping lines for acoustic resonance frequency 4. At other excitation frequencies four or five trapping lines are observed. Different displacement profiles of the transducer can produce different (more or less) trapping lines in the standing waves, with more gradients in the displacement profile generally creating greater trapping forces and more trapping lines.

In the present systems, the system is operated at a voltage such that the particles and particle clusters are trapped in the ultrasonic standing wave. The particles and clusters are collected in well-defined trapping lines. Each trapping line is aligned with the main direction of the acoustic standing wave. Particles and clusters in the trapping lines are separated by half a wavelength. Within each pressure nodal plane of the standing wave, the particles are trapped at very specific points, typically the minima of the acoustic radiation potential. The axial component of the acoustic radiation force drives the particles, with a positive contrast factor, to the pressure nodal planes, whereas particles with a negative contrast factor are driven to the pressure anti-nodal planes. The radial or lateral component of the acoustic radiation force is the force that traps the particles in the standing wave, clumps or clusters them into tightly packed clusters, which then gravity separate when the clusters reach a critical size. In systems using typical transducers, the radial or lateral component of the acoustic radiation force is typically several orders of magnitude smaller than the axial component of the acoustic radiation force. It therefore has two limitations. It has very weak trapping capabilities of particles and moreover, it cannot generate tightly enough packed clusters that will separate out due to gravity. The lateral force in the present devices can be significant, on the same order of magnitude as the axial force component. The strong clustering capability leads to rapid formation of clusters which continuously separate out from the host fluid through gravity/buoyancy separation.

The three-dimensional acoustic standing waves are the result of superposition of the vibration modes of the piezoelectric element. The three dimensional force field results in strong gradients within every nodal plane of the standing wave. Multiple particle clusters are formed along a line in the axial direction of the standing wave, as illustrated in FIG. 9B. For optimum collection, the shape of the particle clusters should give the lowest drag. At particle Reynolds numbers below 20, cylindrical shapes have significantly lower drag coefficients than spheres. Cylinders can also carry significantly more particles (mass) for a given surface area, so that a cylindrical particle cluster will have higher gravity/buoyancy forces and lower resistance drag than a spherical particle cluster. Thus a cylindrical particle cluster will drop out faster than other shapes. It is noted that “cylinder” is used as a shorthand for describing the shape of such clusters, which may perhaps be better described as being ellipsoidal.

Referring back to FIG. 1, the overall system thus operates as follows. One or more acoustic standing waves are created between the transducer 130 and the reflector 140 of the separation unit 120; these waves also pass through the container 110. Particles present in the fluid/particle mixture in the container 110 are trapped in acoustic standing waves at the pressure nodes for particles with positive acoustic contrast and at the pressure anti-nodes for particles with negative acoustic contrast, where they agglomerate, aggregate, clump, or coalesce into larger clusters of particles. The clusters then either rise or sink and are separated from the fluid, which as a result is clarified. Gravity driven flows are present in the system which further enhance the clarification. When clumps of particles settle, an equal volume of lighter and clarified fluid is displaced from the region of the bottom and moves to the top.

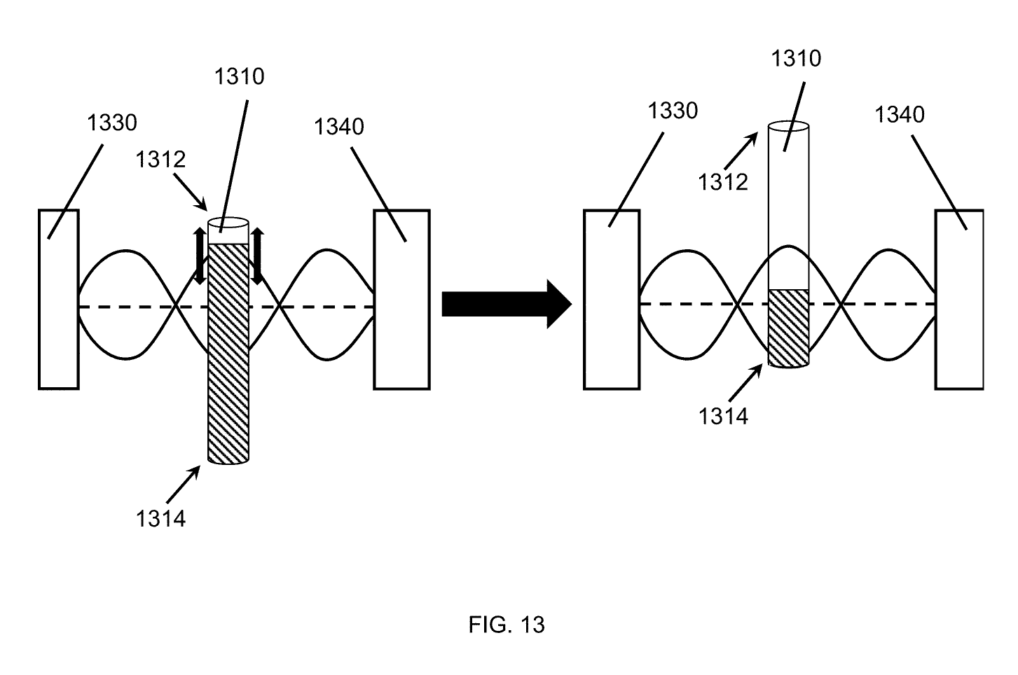

This is also illustrated in FIG. 13. The centrifuge tube 1310 holds a primary fluid and particles therein (e.g., cells dispersed in a buffer solution). An acoustic standing wave (e.g., a multi-dimensional acoustic standing wave) is formed between a transducer 1330 and a reflector 1340. The tube 1310 is drawn through the wave (as indicated by arrows on the left side of FIG. 13). The tube can be drawn upwards to sweep the non-flowing mixture from an upper end 1312 of the tube to a lower end of the tube 1314, concentrating the particles at the lower end of the tube (as shown on the right side of FIG. 13). Alternatively, the tube can be drawn downwards to sweep the non-flowing mixture from the lower end 1314 to the upper end 1312 of the tube. Alternatively, the acoustic standing wave is moved up or down relative to the tube, while the tube is held stationary. This results in a “sweeping effect” through the non-flowing fluid mixture in the tube 1310, which improves the settling or buoyancy of particles that are disposed within the fluid.

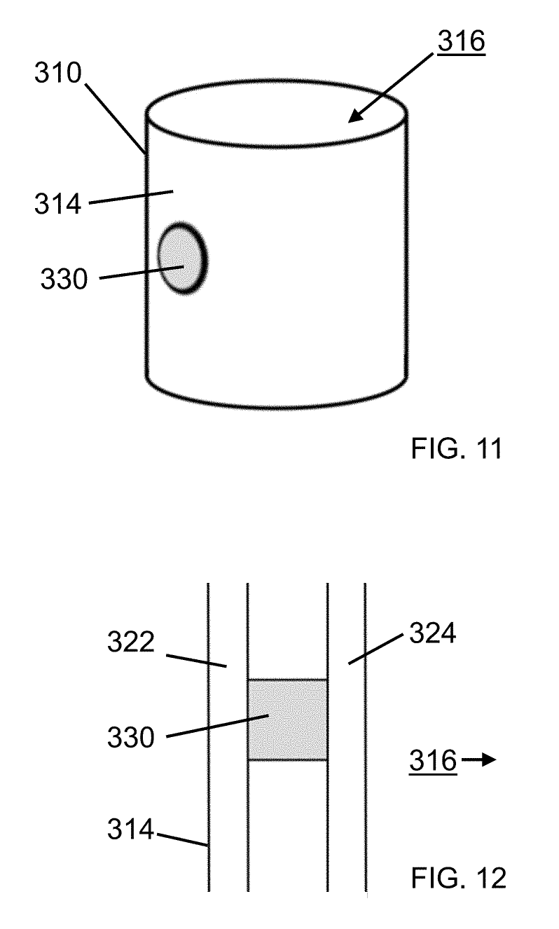

FIG. 11 and FIG. 12 illustrate another embodiment of an acoustophoretic device in accordance with the present disclosure. FIG. 11 is an exterior view of the bag, and FIG. 12 is a cross-sectional view of the bag. In this embodiment, the acoustophoretic device generally includes a disposable separation bag 310. The disposable separation bag 310 includes an exterior surface 314 and an interior volume 316 bounded by the exterior surface 314. The disposable separation bag 310 may be made from at least one polymer layer (e.g., polyethylene, polyurethane, polypropylene, and the like). It is also contemplated that the bag can be made from multiple layers of differentially functioning polymer layers. Those polymer layers may function as a waterproof layer, as a layer that provides strength, etc. For example, in some instances, the exterior (i.e. outermost layer) of the bag is a polyethylene terephthalate (PET) polymer. A middle or central layer of the bag can be typically ethylene vinyl alcohol (EVOH) or polyvinyl acetate (PVA). The interior layer (contacting the bioreactor cell culture medium) is typically a polyethylene polypropylene such as low-density polyethylene or very low density polyethylene. The bag has a large interior volume, generally of at least one liter, up to 1000 liters, and even larger as desired.

An ultrasonic transducer 330 is at least partially disposed inward of the exterior surface 314 of the separation bag 310, such that an acoustic standing wave can be created in the interior volume 316 of the bag. The ultrasonic transducer 330 includes a piezoelectric element driven by a signal to create the acoustic standing wave. As illustrated in FIG. 12, the ultrasonic transducer 330 is between two polymeric layers 322, 324 (please note that the two layers are joined together, and there is no free space between them—this is an artifact of the drawing). The ultrasonic transducer and the acoustic standing wave are the same as described with reference to the various other embodiments disclosed herein. That is, the acoustic standing wave field is created by the ultrasonic transducer 330 within the interior volume 316 of the disposable separation bag 310 such that particles disposed within the fluid can coalesce or agglomerate and drop below or rise above the acoustic standing wave field due to gravitational or buoyancy forces. Put another way, this embodiment allows for a disposable system whereby solids disposed within the fluid in the bag may be agglomerated and drop out of solution above or below the acoustic standing wave field due to gravitational/buoyancy forces and the acoustic standing wave field, thereby resulting in clarification of the fluid in the bag. Here, the reflector is the air that is on the opposite side of the bag from the ultrasonic transducer. This bag is not used with the separation unit 120 of FIG. 1.

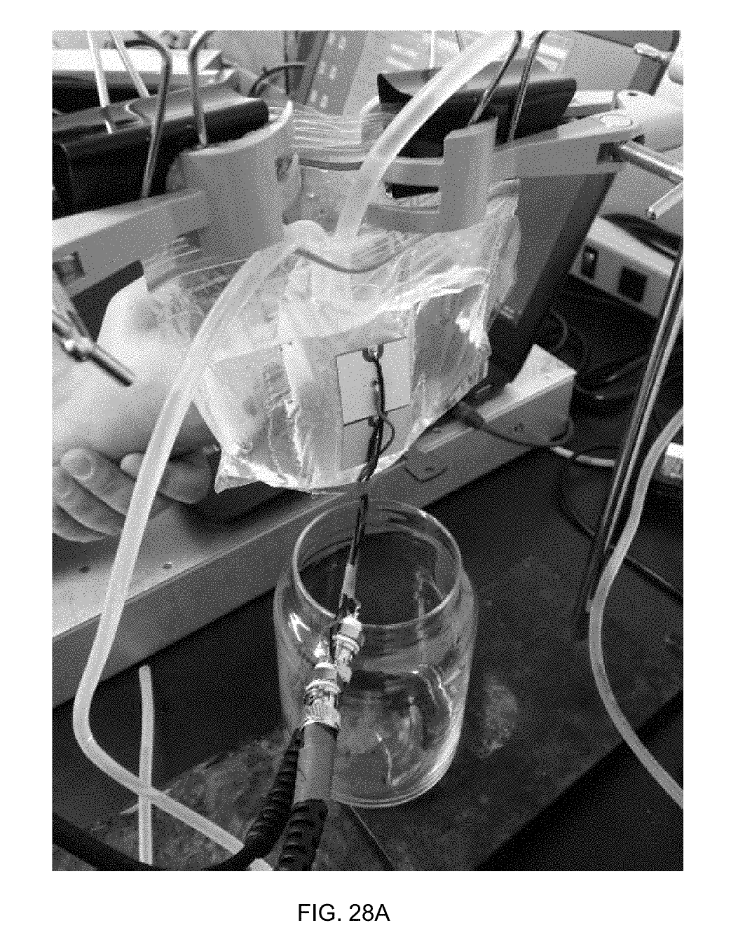

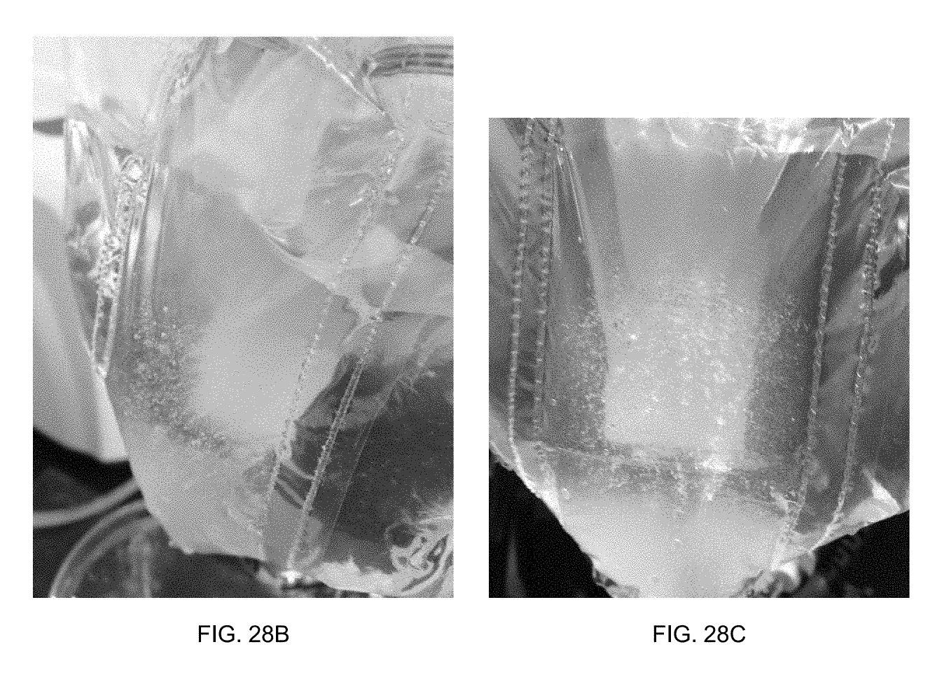

FIG. 28A, FIG. 28B, and FIG. 28C illustrate a separation bag similar to that described in FIG. 12. As seen in these three figures, the separation bag includes at least one piezoelectric transducer affixed thereto, here on the exterior surface of the bag. A smaller bag is placed within the separation bag, the smaller bag containing fluid and cells. The separation bag is filled with a coolant, such as water, between the transducer and the smaller bag. The ultrasonic transducer is operated to cause separation of the cells from the fluid in the smaller bag. The reflector is the air that is on the opposite side of the separation bag from the ultrasonic transducer. Such a reflector is implemented as a pressure release boundary that is formed with the transition of media, e.g., fluid to air. The separation bag or the smaller bag may be formed to have a non-planar or faceted surface opposite the ultrasonic transducer that can operate as a reflector of acoustic energy.

Various types of plastics can be used to form the container of the present disclosure. Matching the impedance value of the plastic chosen is important and will depend upon the frequency at which the ultrasonic transducer(s) is driven to generate the planar, multi-dimensional, or combination acoustic standing wave. The containers or separation bags disclosed herein may be formed of one or more of the materials of Table 1, depending on the desired characteristics of the containers or separation bags and the desired frequency at which the ultrasonic transducer(s) are to be driven for the non-flow separation and clarification of the fluid.

Table 1 below shows the impedance values for various types of plastics. The values in Table 1 are VI=longitudinal sound velocity (m/s); D=density (g/cm³); and Z=acoustic impedance (Megarayls).

TABLE 1
Impedance Values for Various Plastics

| Material | VI | D | Z |
|---|---|---|---|
| ABS | 2,230 | 1.03 | 2.31 |
| Acrylic Plexiglas | 2,750 | 1.19 | 3.26 |
| Adiprene | 1,689 | 1.16 | 1.94 |
| Bakelite | 1,590 | 1.40 | 3.63 |
| Cellulose Butyrate | 2,140 | 1.19 | 2.56 |
| Delrin | 2,430 | 1.42 | 3.45 |
| EPO-TEK 301 | 2,640 | 1.08 | 2.85 |
| Ethyl Vinyl Acetate | 1,800 | 0.94 | 1.69 |
| Neoprene | 1,600 | 1.31 | 2.10 |
| Mylar | 2,450 | 1.18 | 3.00 |
| Nylon 6/6 | 2,600 | 1.12 | 2.90 |
| Polycarbonate | 2,270 | 1.22 | 2.77 |
| Polyester Casting Resin | 2,290 | 1.07 | 2.86 |
| Polyethylene | 1,950 | 0.90 | 1.76 |
| Polyethylene (high-density) | 2,430 | 0.96 | 2.33 |
| Polyethylene (low-density) | 1,950 | 0.92 | 1.79 |
| Polypropylene | 2,470 | 0.88 | 2.40 |
| Polystyrene | 2,320 | 1.04 | 2.42 |
| Polyurethane | 1,700 | 1.04 | 1.80 |
| PVC | 2,380 | 1.38 | 3.27 |
| PVDF | 2,300 | 1.79 | 4.20 |
| Scotch Tape (2.5 mm thick) | 1,900 | 1.16 | 2.08 |
| Vinyl (rigid) | 2,230 | 1.33 | 2.96 |

In biological or other applications, some or all of the parts of the system (e.g., the container, separation unit, etc.) can be separated from each other and be disposable. The techniques and examples of the present disclosure may have advantages over centrifuges and filters by being able to attain better separation of the fluid from particles disposed therein without lowering the viability of the particles.

The transducers may also be driven to create rapid pressure changes to prevent or clear blockages due to agglomeration of the particles. The frequency of the transducers may also be varied to obtain optimal effectiveness for a given power.

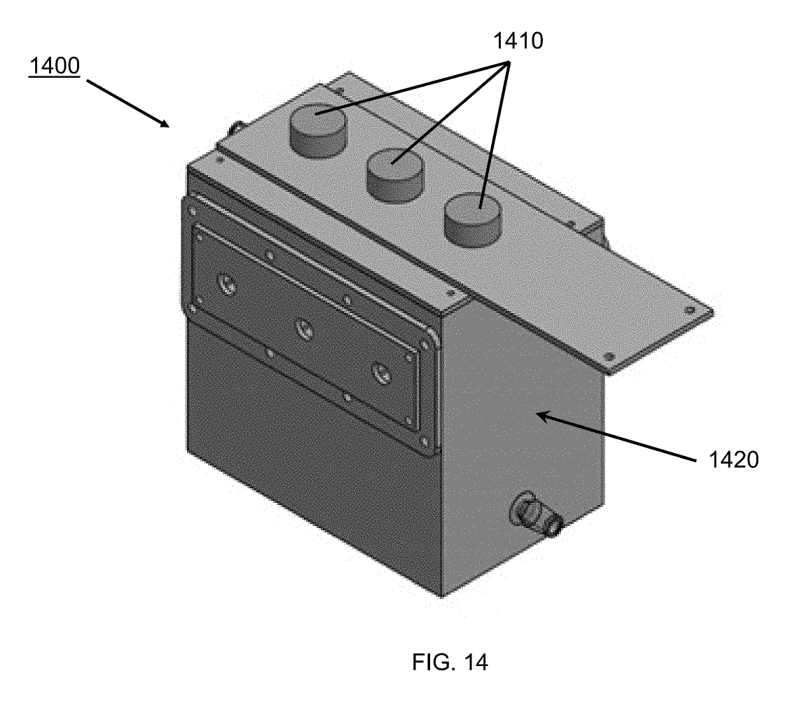

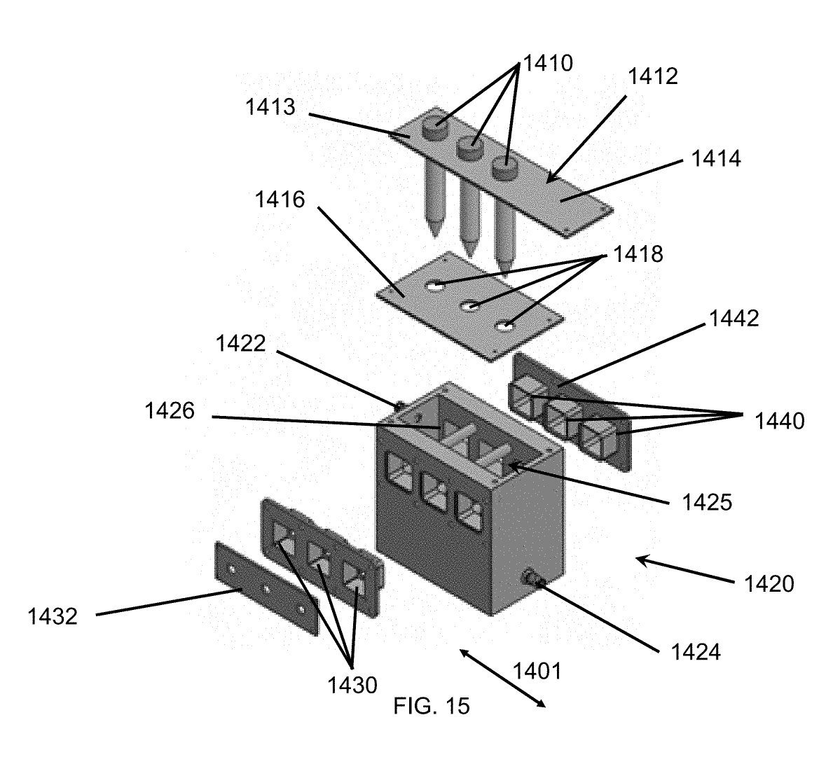

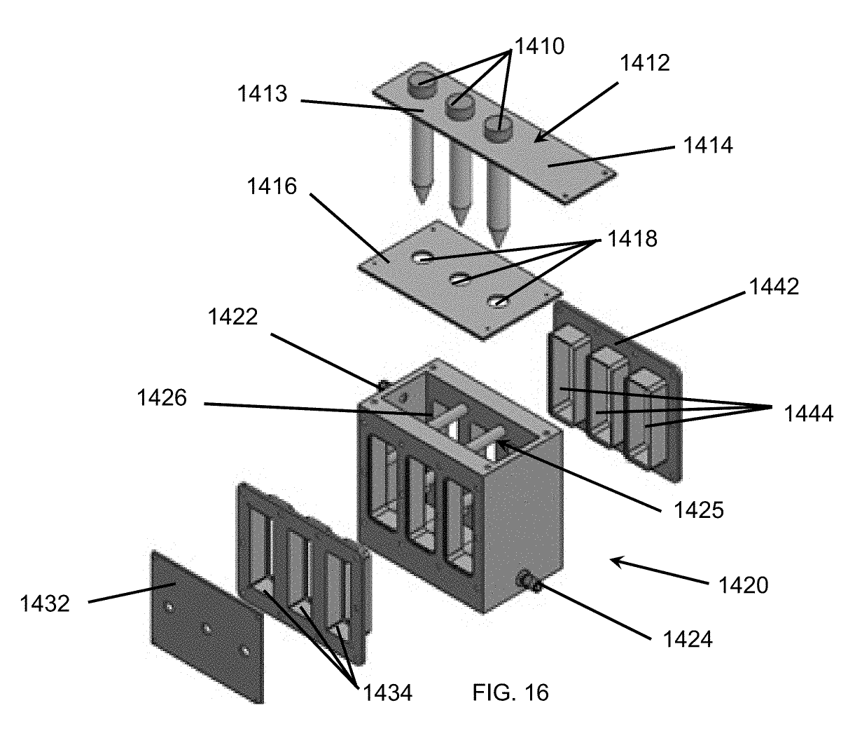

An “acoustic centrifuge system” can be designed to operate with multiple containers such as test tubes or centrifuge tubes. A first example embodiment of such an acoustic centrifuge system 1400 is shown in FIG. 14 and FIG. 15. The system 1400 is an automated system that, very generally, includes a separation unit that can be operated with multiple containers simultaneously. Referring first to FIG. 14, in this example embodiment, the system 1400 includes a separation unit 1420. Three containers 1410, which are test tubes, are illustrated as being used with the separation unit. The test tubes 1410 generally contain a primary fluid and particles therein (e.g., cells dispersed in a buffer solution).

Referring now to FIG. 15, the separation unit 1420 has an interstitial space 1425 therein. A fluid (e.g. water) may be present in interstitial space 1425. The separation unit 1420 includes an inlet 1422 and an outlet 1424 to the interstitial space 1425, such that the fluid can be flowed through the interstitial space (which then acts as a flow chamber).

The separation unit 1420 has a plurality of ultrasonic transducers 1430 arranged along a length of the separation unit (indicated by arrow 1401). Here, three ultrasonic transducers 1430 are illustrated, and the transducers are square transducers. The transducers 1430 are joined together by a transducer plate 1432. The transducers are arranged on a common wall of the separation unit. A plurality of reflectors 1440 are also arranged along a length of the separation unit. The reflectors 1440 are joined together by a reflector plate 1442. The reflectors are also arranged on a common wall of the separation unit, which is across from the wall on which the transducers are mounted. The transducer and reflector plates fix the transducers and reflectors, respectively, to the separation unit.

The separation unit 1420 also includes an alignment plate 1426 along the top 1426 of the separation unit. The alignment plate includes a plurality of apertures 1418 for accessing the interstitial space 1425. Each aperture is aligned with a transducer-reflector pair. Any desired number of transducers and reflectors and apertures may be present in the separation unit, can be used, corresponding for example to the number of containers used in the system.

The system/separation unit 1420 includes a container plate 1412. The container plate includes a main portion 1413 and a flange portion 1414. The main portion includes a plurality of apertures therein, which are shown here as holding containers 1410 (e.g., centrifuge tubes or test tubes). The apertures of the container plate are aligned with the apertures of the alignment plate, such that the containers will be substantially aligned with corresponding transducer-reflector pairs. The flange portion 1414 extends past the walls of the separation unit. The container plate 1413 joins the containers 1410 together, so they can be manipulated together. The flange portion 1414 provides an easy-to-grip area for moving the containers 1410 relative to the separation unit 1420 without having to directly contact the containers, thereby reducing contamination of any samples contained therein. In particular embodiments, an automated device can be used to engage the container plate and move the containers. In particular, a programmed process can be used to automatically move the containers, such that the entire system and process can be automated.

The base and the walls of the separation unit 1420 are generally solid, with holes cut out for the various openings, which can be used to access the interstitial space and operate the device. The separation unit can be 3D-printed or made using conventional techniques, and can be made from plastic or metal.

A second layout for the acoustic centrifuge system 1400 can be seen in FIG. 16. This embodiment is very similar to that of FIG. 15, except that the transducers 1434 and the reflectors 1444 are rectangular, and span substantially the height of the separation unit.

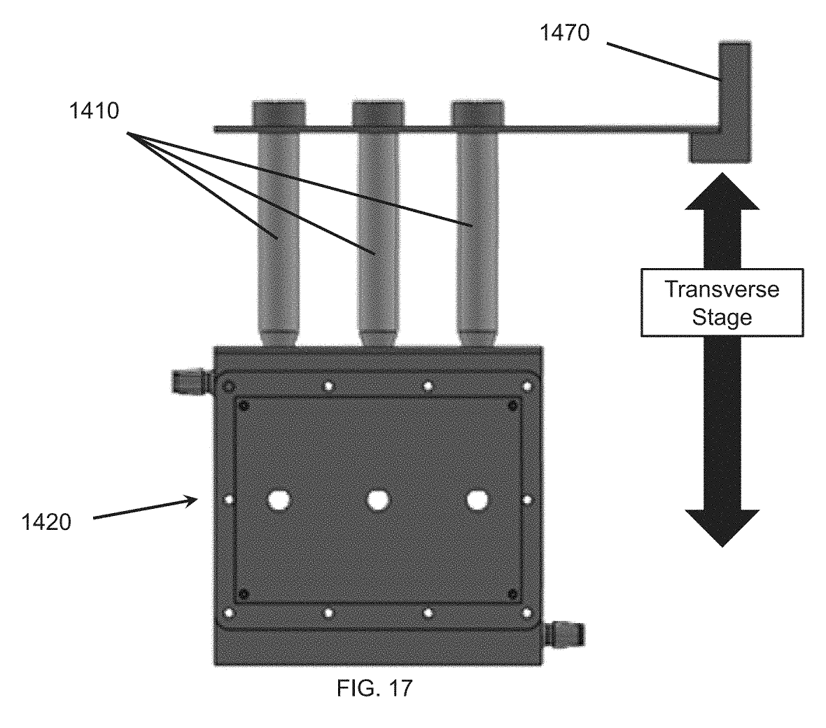

FIG. 17 illustrates a side view of the system. As depicted by the arrow, the containers 1410 can be moved up and down relative to the separation unit 1420 by, for example, the arm 1470 of an automated device. In this way, upward movement of the containers toward an upper end of the separation unit will cause gravitational settling of particles in the containers at the lower ends of the containers, such as is depicted in FIG. 13.

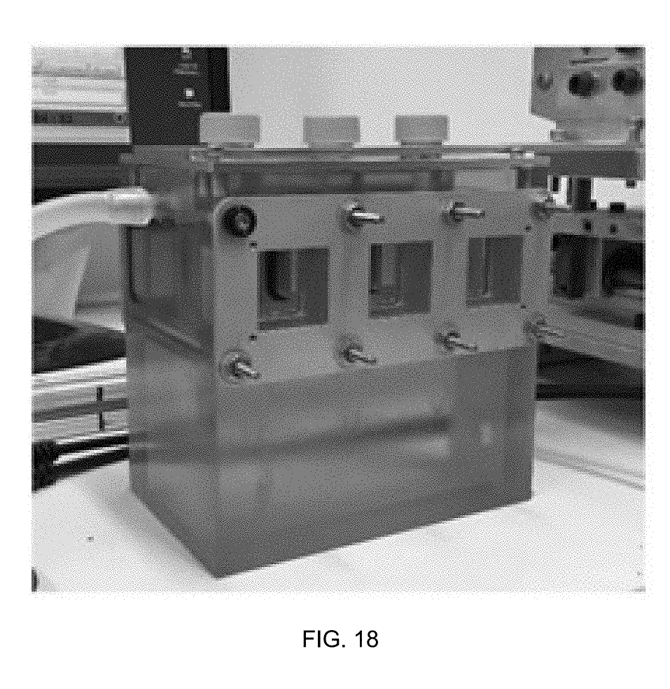

FIG. 18 is a photograph of an acoustic centrifuge system constructed in accordance with the first example system depicted in FIG. 14 and FIG. 15.

As discussed, the containers can be test tubes or centrifuge tubes. They can have a square plan cross-section, or a circular plan cross-section (plan view being along the lengthwise axis of the tubes). The containers can be made of plastic, glass, polycarbonate, low-density polyethylene, or high-density polyethylene, or other material as suitable or desired.

The following examples are provided to illustrate the devices, components, and methods of the present disclosure. The examples are merely illustrative and are not intended to limit the disclosure to the materials, conditions, or process parameters set forth therein.

### EXAMPLES

### Example 1

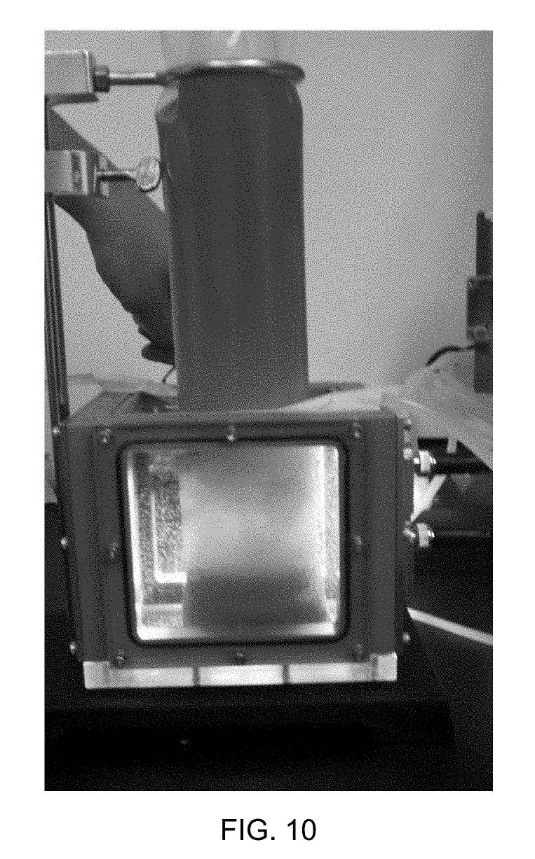

FIG. 10 shows an experimental setup for an acoustophoretic device as described in detail above. This acoustophoretic device is very similar to that illustrated in FIG. 1, except the container is a volumetric plastic bag. The lower end of the plastic bag is disposed within the walls of the separation unit and the separation unit includes an ultrasonic transducer and reflector. The ultrasonic transducer is driven by a voltage signal provided by a BNC cable. An acoustic standing wave is generated by the ultrasonic transducer through the separation unit and lower end of the container for clarifying the fluid contained therein.

The clarification and separation process was conducted where the volumetric plastic bag was filled with a non-flowing fluid medium, namely a fluid and yeast mixture. In accordance with the previously described process, the bag containing the fluid mixture was placed into the separation unit with the bag situated between the transducer and the reflector of the separation unit such that the acoustic standing wave passed through both the plastic bag and the fluid mixture. The fluid contained yeast at a 6% concentration in a 1000 mL volume. The mixture had a starting NTU (Nephelometric Turbidity Units) of 11,800 and, after the above-described separation and clarification process using the devices and components described herein, the upper clarified layer had a final NTU of 856, demonstrating the effectiveness of clarifying a non-flowing mixture of fluid and yeast cells or static environment using acoustophoresis. This evidences that clarification of the fluid is occurring as the yeast cells coalesce or agglomerate in the acoustic standing wave and sink to the lower end of the container due to gravitational forces.

The parameters for the tests are shown in Table 2 below. Table 2 shows clarification results for a 6% yeast solution in water that was separated for 40 minutes using piezoelectric ultrasonic transducers excited at 2.2196 MHz and 2.2147 MHz. The feed is the starting mixture, in which the yeast is dispersed. The permeate is the clarified fluid at the top of the bag, and contains a lower concentration of particulate compared to the feed. The concentrate is the fluid at the bottom of the bag, and contains a higher concentration of particulate compared to the feed.

TABLE 2
Clarification results for a 6% yeast solution in water

| 6% yeast | Volume | Duration | Frequency Top- | Frequency Top- |
|---|---|---|---|---|
| solution in | (mL) | (minutes) | Top (MHZ) | Bottom (MHZ) |
| water | 1,000 | 40 | 2.2196 | 2.2147 |
| | NTU | NTU | NTU | PCV |
| | Feed | Permeate | Concentrate | Concentrate |
| | 11,800 | 856 | 100,200 | — |

### Example 2

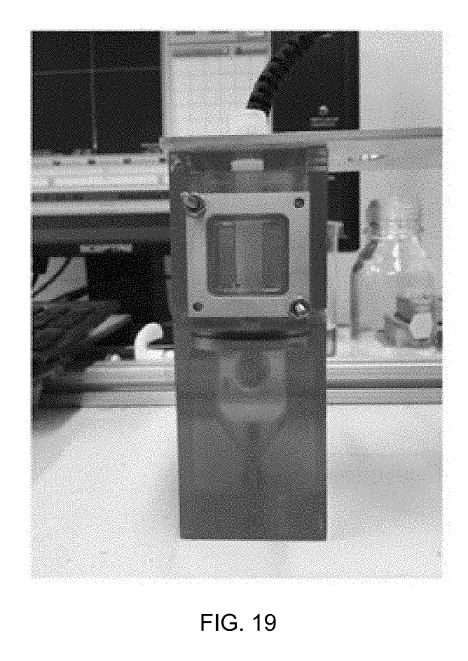

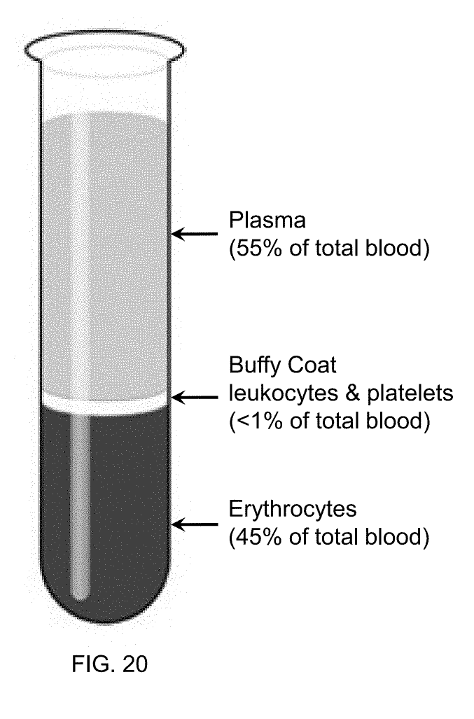

FIG. 19 is a photograph of an acoustic centrifuge system including a single transducer and reflector in the separation unit. This system can be used, for example, to separate the constituent components of blood in a single centrifuge tube, such as is shown in FIG. 20. As noted here, the various components of blood can be separated by density. The densest components are erythrocytes, which make up 45% of the volume of blood. The next densest components in the buffy coat, which contains leukocytes and platelets, and make up less than 1% of blood volume. The least dense component of blood is the plasma, which makes up 55% of blood volume.

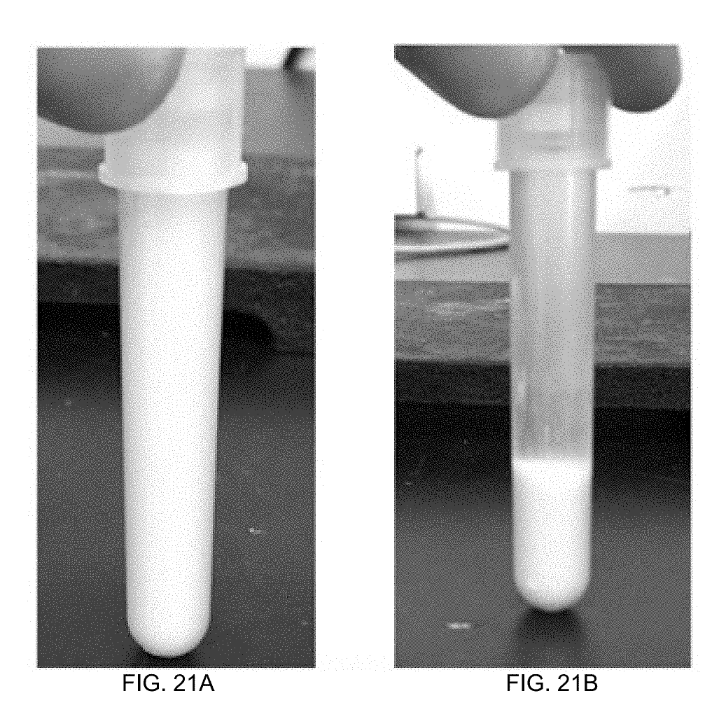

The device of FIG. 19 was used for testing. A 16% yeast suspension was placed into a centrifuge tube. A 1-inch×1-inch PZT-8 transducer was used in the separation chamber and run at approximately 2 MHz, drawing 8 watts, with an amplitude of 80 volts. FIG. 21A depicts the yeast solution in the centrifuge tube prior to being subjected to the acoustic centrifuge process. After performing the acoustic centrifuge process for approximately five minutes, 99.7% of the yeast particles were found to have formed a pellet/cluster at the lower end of the centrifuge tube, as shown in FIG. 21B.

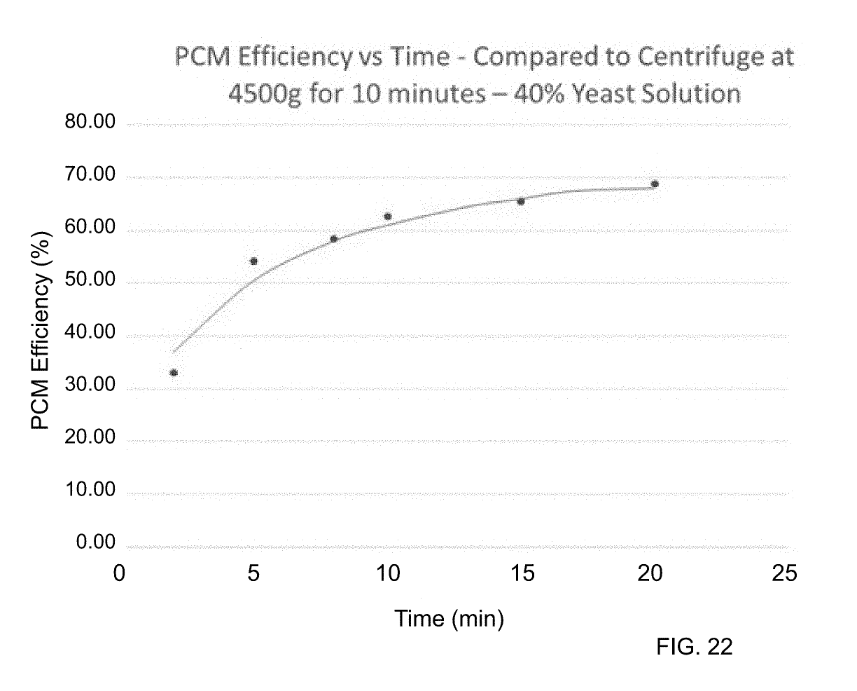

FIG. 22 is a graph of the pack cell mass (PCM) efficiency versus time for a 40% yeast solution. The acoustic centrifuge process of the present disclosure using a round tube as the container was compared to a centrifuge at 4500 g for 10 minutes. After about 20 minutes of processing, a PCM efficiency of about 70% was achieved.

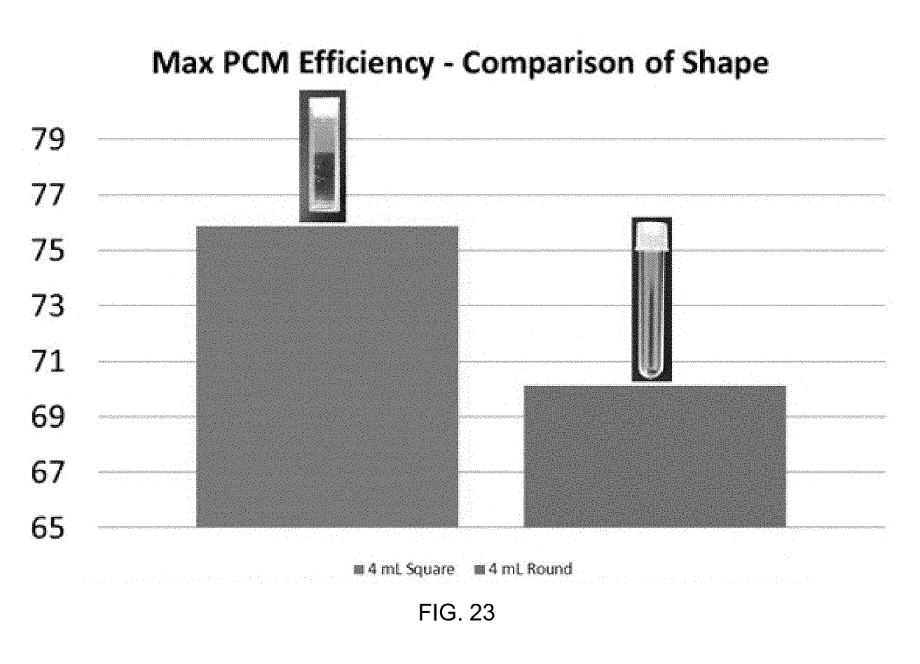

FIG. 23 is a graph that compares the effect of container shape on maximum PCM efficiency. The left bar represents a four milliliter (mL) square tube, and the right bar represents a four mL round tube. The square tube exhibited a better maximum PCM efficiency than the round tube (approximately 76% versus 70%). The container shape had a significant effect on maximum PCM efficiency.

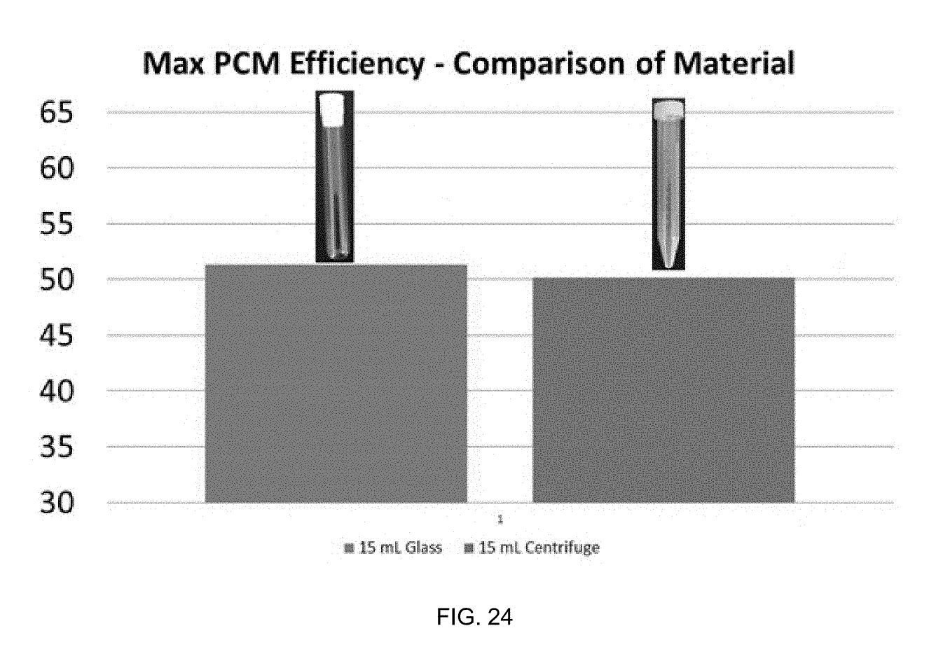

FIG. 24 graphically compares the effect of container material on maximum PCM efficiency. The left bar represents a 15 mL glass container, and the right bar represents a 15 mL polyethylene centrifuge tube. The containers exhibited similar PCM efficiencies, with the glass container being slightly better (51% versus 50%).

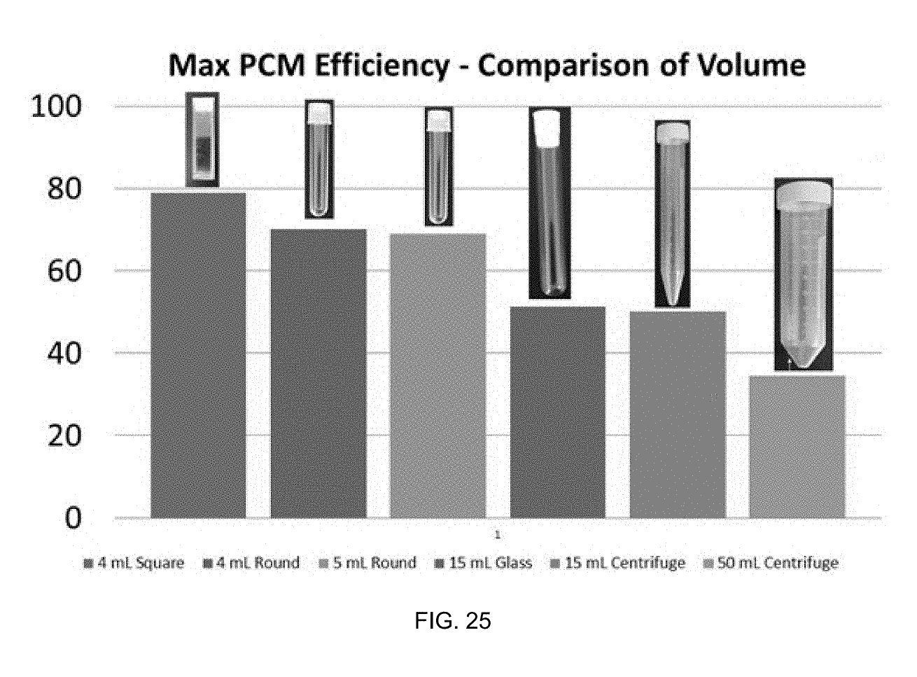

Finally, FIG. 25 graphically compares the effect of container volume on maximum PCM efficiency. Six tubes of different volumes, shapes, and materials were used. The leftmost bar represents a four mL square glass container; the second bar from the left represents a four mL round glass container; the third bar from the left represents a five mL round glass container; the third bar from the right represents a 15 mL glass container; the second bar from the right represents a 15 mL polyethylene centrifuge tube; and the rightmost bar represents a 50 mL polyethylene centrifuge tube. As can be seen in FIG. 25, as the container volume was increased, the maximum PCM efficiency decreased.

This testing thus showed that the acoustic centrifuge process of the present disclosure was capable of concentrating and separating material using acoustophoresis coupled with a container (e.g., a centrifuge tube or test tube). In particular, after just five minutes of processing, 99.7% of the yeast particles were found to have formed a pellet/cluster at the lower end of the centrifuge tube, as shown in FIG. 21B. Further, testing resulted in a PCM efficiency up to 75% that was found to improve with a lower container volume, upward movement of the container relative to the separation unit, the creation of a pellet/cluster in the acoustic filed, and the use of rectangular containers. This confirms that the acoustic centrifuge process of the present disclosure is as capable as or better than traditional centrifugal processes for concentration and separation of material. In particular, the acoustic centrifuge systems and processes of the present disclosure are smaller and use fewer mechanical parts, thus making them less expensive to manufacture than traditional centrifuges. The acoustic centrifuge systems and processes of the present disclosure also result in less packing and forces on the cells, and can be operated in a continuous process with the opportunity to handle more samples than a conventional centrifuge.

### Example 3

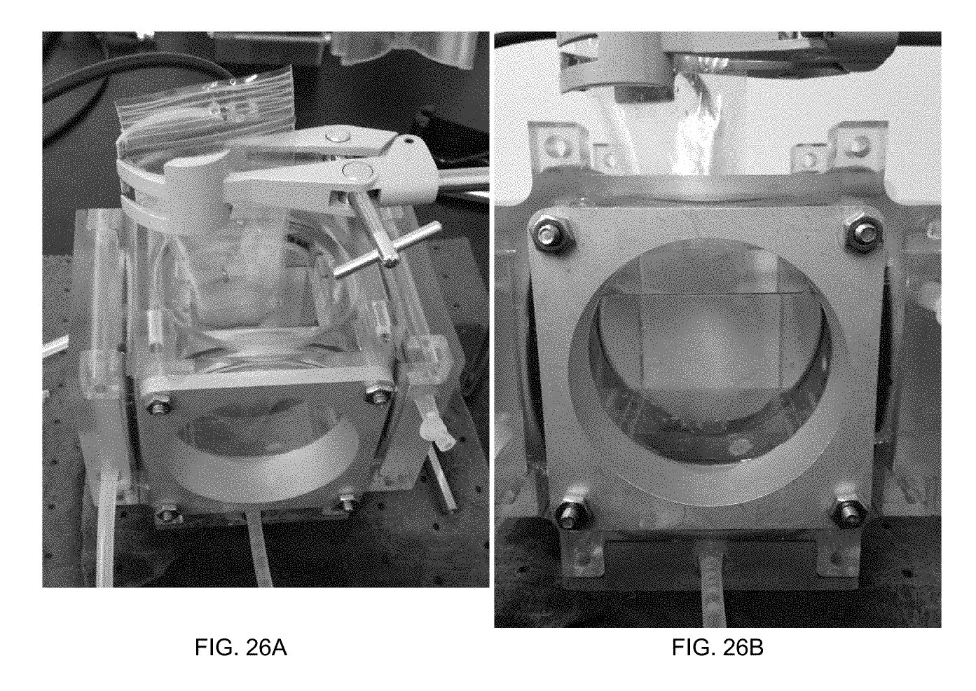

A separation unit with an acoustic chamber of volume 1 inch×1 inch×1 inch with a faceted reflector and a piezoelectric transducer array of 9×9 elements was used to create separation in a bag. Two pictures of this setup are provided as FIG. 26A and FIG. 26B.

The bag contained a mixture of T-cells and fluid, with a total feed volume of 20.312 mL and a feed cell density of 1.08×10⁶ (1.08E6) cells/mL, for a total of 21.94E6 cells. To cause separation, the piezoelectric transducers were operated at 30 W for 20 minutes. After being turned off, the bag was allowed to sit for 10 minutes to permit the separated cells to settle down.

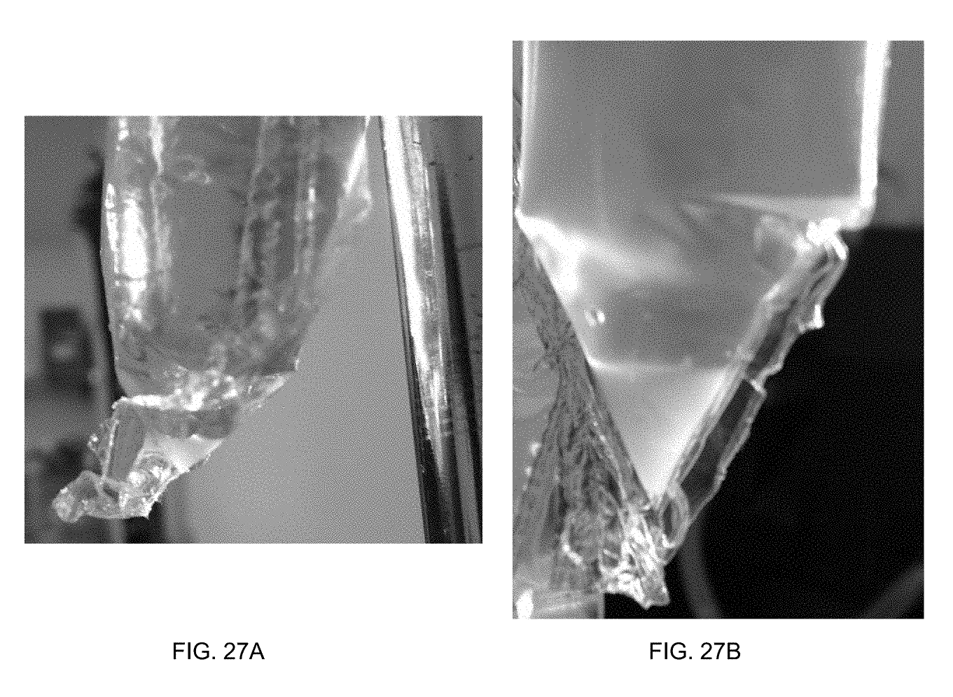

FIG. 27A and FIG. 27B are two separate pictures of the bag after sitting. As seen here, the bag is tapered, and a concentrated pallet has formed at the tip of the bag after application of acoustic separation.

Next, fluid was removed from the top of the bag. First, 14 mL was removed from the bag at once. Smaller amounts of fluid were then removed. The cell density of the removed fluid was measured using a Vi-CELL™ Cell Counter. This permitted calculation of the number of cells in the fluid remaining in the bag, and the cell density of the fluid remaining in the bag.

The results are listed in Table 3. The first column indicates the amount of fluid removed from the bag (mL). The second column indicates the amount of fluid remaining in the bag (mL). This shows how cell density varies along the depth of the bag. The third column is the cell density (million cells/mL) measured in the fluid removed from the bag. The fourth column is the total number of cells in the fluid removed from the bag (in millions of cells). The fifth column is the total number of cells remaining in the bag (×1E6 cells) after the fluid is removed in the bag. The sixth column is the volume concentration factor, or the starting volume divided by the remaining volume. The seventh column is the percentage of initial cells left in the bag after the fluid has been removed, and is a parameter that indicates the efficiency of the system.

TABLE 3

| | | | | Total | | |
|---|---|---|---|---|---|---|
| | | | Total Cells | Cells | | |
| | | Cell | in | left in | | % |
| Volume | Remaining | Density of | Removed | the | | Cells |
| removed | volume in | Removed | Fluid | bag | Volume | left in |
| from the | the bag | Fluid | (×1E6 | (×1E6 | Concentration | the |
| bag (mL) | (mL) | (×1E6 cells/mL) | cells) | cells) | Factor | bag |
| 14 | | | | | | |
| 1 | 5.312 | 0.05 | 0.05 | 21.31 | 3.82 | 97.48 |
| 1 | 4.312 | 0.05 | 0.05 | 21.26 | 4.71 | 97.25 |
| 1 | 3.312 | 0.05 | 0.05 | 21.21 | 6.13 | 97.03 |
| 0.6 | 2.712 | 0.07 | 0.04 | 21.17 | 7.49 | 96.86 |
| 0.6 | 2.112 | 0.05 | 0.03 | 21.15 | 9.62 | 96.73 |
| 0.6 | 1.512 | 0.09 | 0.06 | 21.09 | 13.43 | 96.48 |
| 0.6 | 0.912 | 0.14 | 0.08 | 21.01 | 22.27 | 96.10 |
| 0.3 | 0.612 | 0.06 | 0.02 | 20.99 | 33.19 | 96.01 |
| 0.3 | 0.312 | 0.22 | 0.07 | 20.92 | 65.10 | 95.71 |
| 0.25 | 0.062 | 2.12 | 0.53 | 20.39 | 327.61 | 93.29 |

As indicated at the end of Table 3, the final 0.062 mL remaining in the bag contained 20.39E6 cells, for a final cell density of 328.9E6 cells/mL. About 93% of the original cells were recovered.

The experiment was performed a second time in the same manner, but with a higher feed cell density. In the second experiment, the bag had a total feed volume of 23.41 mL and a feed cell density of 11.59E6 cells/mL, for a total of 271.3E6 cells. The results are shown in Table 4.

TABLE 4

| | | | Total Cells | Total | | |
|---|---|---|---|---|---|---|
| | | Cell | in | Cells | | % |
| Volume | Remaining | Density of | Removed | left in | | Cells |
| removed | volume in | Removed | Fluid | the bag | Volume | left in |
| from the | the bag | Fluid | (×1E6 | (×1E6 | Concentration | the |
| bag (mL) | (mL) | (×1E6 cells/mL) | cells) | cells) | Factor | bag |
| 15 | | | | | | |
| 1 | 7.41 | 1.89 | 1.89 | 267.12 | 3.16 | 98.41 |
| 1 | 6.41 | 1.83 | 1.83 | 265.29 | 3.65 | 97.74 |
| 1 | 5.41 | 1.79 | 1.79 | 263.50 | 4.33 | 97.08 |
| 1 | 4.41 | 2.00 | 2.00 | 261.50 | 5.31 | 96.34 |
| 1 | 3.41 | 2.30 | 2.30 | 259.20 | 6.87 | 95.49 |
| 1 | 2.41 | 2.52 | 2.52 | 256.69 | 9.71 | 94.57 |
| 1 | 1.41 | 4.71 | 4.71 | 251.97 | 16.60 | 92.83 |
| 1 | 0.41 | 7.00 | 7.00 | 244.97 | 57.10 | 90.25 |

As indicated at the end of Table 4, the final 0.41 mL remaining in the bag contained 244.97E6 cells, for a final cell density of 597.5E6 cells/mL. About 90% of the original cells were recovered.

The present disclosure has been described with reference to exemplary embodiments. Modifications and alterations may occur to others upon reading and understanding the preceding detailed description. It is intended that the present disclosure be construed as including all such modifications and alterations insofar as they come within the scope of the appended claims or the equivalents thereof.

1. An acoustic centrifuge system for clarifying a discrete volume of fluid in each of a plurality of containers, the system comprising:
a separation unit having an interstitial space therein, and comprising:
a plurality of ultrasonic transducers arranged along a length of the separation unit, each transducer including a piezoelectric material configured to be driven to create an acoustic standing wave within the interstitial space; and
an alignment plate along a top of the separation unit, the alignment plate having a plurality of apertures, each aperture being aligned with one of the ultrasonic transducers.

2. The system of claim 1, wherein the separation unit further comprises a transducer plate joining the plurality of ultrasonic transducers such that the plurality of ultrasonic transducers are arranged on a common wall of the separation unit.

3. The system of claim 2, wherein the separation unit further comprises a reflector plate joining a plurality of reflectors together, the reflector plate located across from the transducer plate on an opposite wall of the separation unit.

4. The system of claim 1, wherein each ultrasonic transducer is a square transducer or a rectangular transducer.

5. The system of claim 1, wherein each ultrasonic transducer creates a planar one-dimensional acoustic standing wave.

6. The system of claim 1, wherein each ultrasonic transducer creates a multi-dimensional acoustic standing wave.

7. The system of claim 6, wherein the multi-dimensional acoustic standing wave results in an acoustic radiation force having an axial force component and a lateral force component that are of the same order of magnitude.

8. The system of claim 1, further comprising a container plate that moves relative to the separation unit, the container plate comprising a main portion and a flange portion, the main portion having a plurality of apertures therein, and the flange portion extending outwardly beyond the separation unit.

9. The system of claim 1, further comprising an inlet and an outlet for flowing a fluid through the interstitial space.

10. The system of claim 1, further comprising a plurality of containers.

11. The system of claim 10, wherein each container has a square plan cross-section or a circular plan cross-section.

12. The system of claim 10, wherein each container is made of plastic, glass, polycarbonate, low-density polyethylene, or high-density polyethylene.

13. A method for clarifying non-flowing fluid mixtures in a plurality of different containers, comprising:
receiving a separation unit that has an interstitial space therein, and comprises:
a plurality of ultrasonic transducers arranged along a length of the separation unit, each transducer including a piezoelectric material configured to be driven to create an acoustic standing wave within the interstitial space; and
an alignment plate along a top of the separation unit, the alignment plate having a plurality of apertures, each aperture being aligned with one of the ultrasonic transducers;
placing a plurality of substantially acoustically transparent containers through the apertures of the alignment plate, each container containing a non-flowing mixture of a host fluid and a second fluid or particulate; and
driving each ultrasonic transducer to create the acoustic standing wave in a corresponding container, such that the second fluid or particulate in the corresponding container is trapped in the acoustic standing wave, clumps, clusters, agglomerates, or coalesces together, and continuously rises or settles out of the host fluid due to buoyancy or gravity forces.

14. The method of claim 13, further comprising moving the plurality of containers relative to the separation unit to sweep the non-flowing mixture from an upper end of the container to a lower end of the container.

15. The method of claim 13, further comprising moving the plurality of containers relative to the separation unit to sweep the non-flowing mixture from a lower end of the container to an upper end of the container.

16. The method of claim 13, wherein the plurality of substantially acoustically transparent containers is placed through apertures in a container plate prior to placing the plurality of containers through the apertures of the alignment plate.

17. The method of claim 16, wherein further comprising moving the plurality of containers relative to the separation unit by moving the container plate.

18. The method of claim 17, wherein the container plate is moved using an automated device.

19. The method of claim 13, wherein the frequency of the acoustic standing wave created by each transducer is from about 500 kHz to about 10 MHz.

20. An acoustophoretic bag for clarifying a non-flowing fluid, comprising:
a bag including an interior volume; and
an ultrasonic transducer on the bag, the ultrasonic transducer having a piezoelectric material driven by a voltage signal to create an acoustic standing wave within the interior volume of the bag.

21. The acoustophoretic bag of claim 20, wherein the surface opposite the transducer is embossed.
